(19) United States
(12) Patent Application Publication
(10) Pub. No.: US 2007/0131879 A1
(43) Pub. Date: Jun. 14, 2007

(54) Force provider with adjustable force characteristics for a stage assembly

(75) Inventors: Alex Ka Tim Poon, San Ramon, CA (US); Leonard Wai Fung Kho, San Francisco, CA (US)

Correspondence Address: THE LAW OFFICE OF STEVEN G ROEDER, 5560 CHELSEA AVE, LA JOLLA, CA, 92037, US

(73) Assignee: Nikon Corporation, Tokyo (JP)

(21) Appl. No.: 11/655,578

(22) Filed: Jan. 19, 2007

Related U.S. Application Data
(63) Continuation-in-part of application No. 10/770,873, filed on Feb. 2, 2004.

(51) Int. Cl. A61N 5/00

(52) U.S. Cl. 250/492.2

(57) ABSTRACT

A stage assembly (220A) includes a stage (206A) and a force provider (232A) that provides an acceleration/deceleration force on the stage (206A). The force provider (232A) includes a provider housing (238A) and a piston assembly (240A). The provider housing (238A) defines a piston chamber (344), and includes a first beam aperture (352F), a first cylinder aperture (350F) that is in fluid communication with a fluid at a first pressure and a spaced apart second cylinder aperture (350S) that is in fluid communication with a fluid at the first pressure. The piston assembly (340) includes a piston (356) positioned in the piston chamber (344), and a first beam (358F)) extending through the first beam aperture (352F). The first beam (358F)) is secured to the piston (356). The piston (356) moves along a piston path (462). At a second piston region (462S) of the piston path (462) the piston (356) is positioned between the cylinder apertures (350F)(350S). A control assembly (933) controls the characteristics of the acceleration/deceleration force such as the ramping characteristics of the acceleration/deceleration force and the magnitude of the acceleration/deceleration force.

### RELATED INVENTIONS

This application is a continuation-in-part of application Ser. No. 10/770,873, filed Feb. 2, 2004, which is currently pending. As far as permitted, the contents of application Ser. No. 10/770,873 are incorporated herein by reference.

### BACKGROUND

Exposure apparatuses for semiconductor processing are commonly used to transfer images from a reticle onto a semiconductor wafer during semiconductor processing. A typical exposure apparatus includes an illumination source, a reticle stage assembly that positions a reticle, an optical assembly, a wafer stage assembly that positions a semiconductor wafer, a measurement system, and a control system.

Typically, the wafer stage assembly includes a wafer stage that retains the wafer, and a wafer mover assembly that moves the wafer stage and the wafer. Similarly, the reticle stage assembly includes a reticle stage that retains the reticle, and a reticle mover assembly that moves the reticle stage and the reticle. The rapid acceleration and deceleration rates of the wafer stage and the reticle stage allows for the rapid manufacturing of wafers.

One way to increase acceleration and deceleration of a stage includes utilizing relatively large motors in each stage mover assembly. Unfortunately, the relatively large motors generate heat and consume relatively large amounts of energy.

### SUMMARY

The present invention is directed to force provider assembly including a provider housing and a piston assembly for a stage assembly. The provider housing defines a piston chamber, and includes a first beam aperture, a second beam aperture, a first cylinder aperture and a spaced apart second cylinder aperture. A pressure control assembly is in fluid communication with the cylinder apertures and controls the pressure in the cylinder apertures so that the pressure at each cylinder aperture is approximately the same. In this embodiment, the piston assembly includes a piston positioned in the piston chamber, a first beam extending through the first beam aperture and a second beam extending through the second beam aperture. Each beam is secured to an opposite side of the piston. The piston moves relative to the provider housing within the piston chamber along a piston path. In certain embodiments, the pressure control assembly can be used to adjust the force profile of the force provider assembly.

In one embodiment, the piston path includes a first piston region, a second piston region and a third piston region. When the piston is in the first piston region, the pressure of the fluid on a first piston side of the piston is greater than the pressure of the fluid on a second piston side of the piston. In the first piston region, the piston is positioned between the first beam aperture and the first cylinder aperture. In the second piston region, the pressure on each side of the piston is the same. In the third piston region, the pressure of the fluid on the second piston side is greater than the pressure of the fluid on the first piston side.

In yet another embodiment, the force provider assembly includes a maximum pressure control assembly that is in fluid communication with the provider housing. The maximum pressure control assembly controls the maximum force that is generated by the force provider assembly.

The present invention is also directed to (i) a stage assembly including the force provider, (ii) an exposure apparatus including the stage assembly, and (iii) an object or wafer on which an image has been formed by the exposure apparatus. Further, the present invention is also directed to (i) a method for accelerating and decelerating a stage, (ii) a method for making a stage assembly, (iii) a method for manufacturing an exposure apparatus, and (iv) a method for manufacturing an object or a wafer.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic illustration of an exposure apparatus having features of the present invention;

FIG. 2A is a perspective view of one embodiment of a stage assembly having features of the present invention;

FIG. 2B is a perspective view of another embodiment of a stage assembly having features of the present invention;

FIG. 3A is a perspective view of a force provider assembly having features of the present invention;

FIG. 3B is a cut-away view taken on line 3B-3B of FIG. 3A;

FIG. 4A is a cut-away view of a force provider and a mover secured to a stage in a first stage region and a fluid source;

FIG. 4B is a cut-away of the force provider and the mover secured to a stage in a second stage region and the fluid source;

FIG. 4C is a cut-away of the force provider and the mover secured to a stage in a third stage region and the fluid source;

FIG. 5A is a graph that illustrates position of the stage versus time during movement of the stage;

FIG. 5B is a graph that illustrates velocity of the stage versus time during movement of the stage;

FIG. 5C is a graph that illustrates acceleration of the stage versus time during movement of the stage;

FIG. 5D is a graph that illustrates pressure on a piston versus time during movement of the stage;

FIG. 5E is a graph that illustrates force on the piston versus time during movement of the stage;

FIG. 6A is a perspective view of another embodiment of a force provider assembly having features of the present invention;

FIG. 6B is a cut-away view taken on line 6B-6B of FIG. 6A;

FIG. 7A is a perspective view of yet another embodiment of a force provider assembly having features of the present invention;

FIG. 7B is a cut-away view taken on line 7B-7B of FIG. 7A;

FIG. 7C is a cut-away view of a force provider and a mover secured to a stage approaching a first stage region and a fluid source;

FIG. 7D is a cut-away of the force provider and the mover secured to a stage in the first stage region and the fluid source;

FIG. 7E is a cut-away of the force provider and the mover secured to a stage in the first stage region and the fluid source;

FIG. 7F is a cut-away view of a force provider and a mover secured to a stage in a second stage region and the fluid source;

FIG. 7G is a cut-away of the force provider and the mover secured to a stage in a third stage region and the fluid source;

FIG. 7H is a cut-away view of yet another embodiment of a force provider having features of the present invention;

FIG. 8A is a graph that illustrates the relationship of pressure versus time for different set pressures;

FIG. 8B is a graph that illustrates the relationship of pressure versus time for different piston gaps;

FIG. 9A is a perspective view of still another embodiment of a force provider assembly having features of the present invention;

FIG. 9B is a cut-away view of the force provider assembly of FIG. 9A and a mover secured to a stage in a first stage region;

FIG. 9C is a cut-away view of the force provider assembly of FIG. 9A and the mover secured to the stage in the second stage region;

FIG. 9D is a cut-away view of the force provider assembly of FIG. 9A the mover secured to the stage in a third stage region;

FIG. 9E is a graph that illustrates the resultant pressure versus time;

FIG. 9F is another graph that illustrates the resultant pressure versus time;

FIG. 10A is a cut-away view of yet another embodiment of a force provider assembly with a piston in a first piston region;

FIG. 10B is a cut-away view of the force provider assembly of FIG. 10A with the piston in a second piston region;

FIG. 10C is a cut-away view of the force provider assembly of FIG. 10A with the piston in a third piston region;

FIG. 11A is a flow chart that outlines a process for manufacturing a device in accordance with the present invention; and

FIG. 11B is a flow chart that outlines device processing in more detail.

### DESCRIPTION

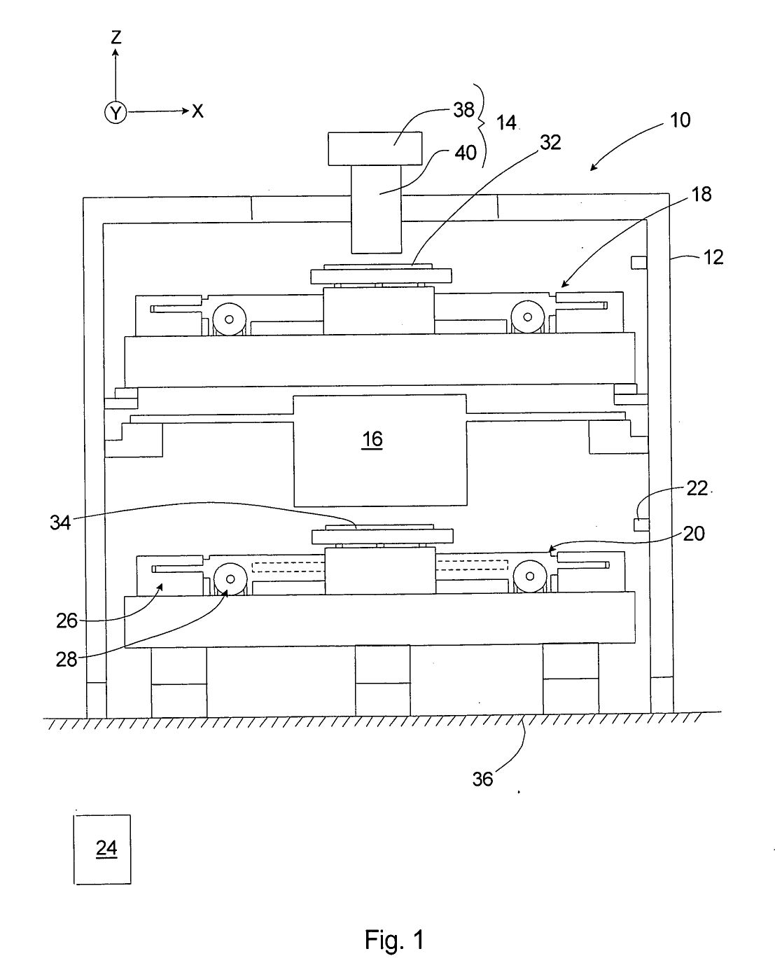

FIG. 1 is a schematic illustration of a precision assembly, namely an exposure apparatus 10 having features of the present invention. The exposure apparatus 10 includes an apparatus frame 12, an illumination system 14 (irradiation apparatus), an optical assembly 16, a reticle stage assembly 18, a wafer stage assembly 20, a measurement system 22, and a control system 24. The design of the components of the exposure apparatus 10 can be varied to suit the design requirements of the exposure apparatus 10. As provided herein, one or both of the stage assemblies 18, 20 can include a stage mover assembly 26 having one or more force provider assemblies 28.

A number of Figures include an orientation system that illustrates an X axis, a Y axis that is orthogonal to the X axis and a Z axis that is orthogonal to the X and Y axes. It should be noted that these axes can also be referred to as the first, second and third axes.

The exposure apparatus 10 is particularly useful as a lithographic device that transfers a pattern (not shown) of an integrated circuit from a reticle 32 onto a semiconductor wafer 34. The exposure apparatus 10 mounts to a mounting base 36, e.g., the ground, a base, or floor or some other supporting structure.

There are a number of different types of lithographic devices. For example, the exposure apparatus 10 can be used as a scanning type photolithography system that exposes the pattern from the reticle 32 onto the wafer 34 with the reticle 32 and the wafer 34 moving synchronously. In a scanning type lithographic device, the reticle 32 is moved perpendicularly to an optical axis of the optical assembly 16 by the reticle stage assembly 18 and the wafer 34 is moved perpendicularly to the optical axis of the optical assembly 16 by the wafer stage assembly 20. Scanning of the reticle 32 and the wafer 34 occurs while the reticle 32 and the wafer 34 are moving synchronously.

Alternatively, the exposure apparatus 10 can be a step-and-repeat type photolithography system that exposes the reticle 32 while the reticle 32 and the wafer 34 are stationary. In the step and repeat process, the wafer 34 is in a constant position relative to the reticle 32 and the optical assembly 16 during the exposure of an individual field. Subsequently, between consecutive exposure steps, the wafer 34 is consecutively moved with the wafer stage assembly 20 perpendicularly to the optical axis of the optical assembly 16 so that the next field of the wafer 34 is brought into position relative to the optical assembly 16 and the reticle 32 for exposure. Following this process, the images on the reticle 32 are sequentially exposed onto the fields of the wafer 34, and then the next field of the wafer 34 is brought into position relative to the optical assembly 16 and the reticle 32.

However, the use of the exposure apparatus 10 provided herein is not limited to a photolithography system for semiconductor manufacturing. The exposure apparatus 10, for example, can be used as an LCD photolithography system that exposes a liquid crystal display device pattern onto a rectangular glass plate or a photolithography system for manufacturing a thin film magnetic head. Further, the present invention can also be applied to a proximity photolithography system that exposes a mask pattern from a mask to a substrate with the mask located close to the substrate without the use of a lens assembly.

The apparatus frame 12 is rigid and supports the components of the exposure apparatus 10. The apparatus frame 12 illustrated in FIG. 1 supports the reticle stage assembly 18, the optical assembly 16 and the illumination system 14 above the mounting base 36.

The illumination system 14 includes an illumination source 38 and an illumination optical assembly 40. The illumination source 38 emits a beam (irradiation) of light energy. The illumination optical assembly 40 guides the beam of light energy from the illumination source 38 to the optical assembly 16. The beam illuminates selectively different portions of the reticle 32 and exposes the wafer 34. In FIG. 1, the illumination source 38 is illustrated as being supported above the reticle stage assembly 18. Typically, however, the illumination source 38 is secured to one of the sides of the apparatus frame 12 and the energy beam from the illumination source 38 is directed to above the reticle stage assembly 18 with the illumination optical assembly 40.

The illumination source 38 can be a g-line source (436 nm), an i-line source (365 nm), a KrF excimer laser (248 nm), an ArF excimer laser (193 nm) or a F₂ laser (157 nm). Alternatively, the illumination source 38 can generate charged particle beams such as an x-ray or an electron beam. For instance, in the case where an electron beam is used, thermionic emission type lanthanum hexaboride (LaB₆) or tantalum (Ta) can be used as a cathode for an electron gun. Furthermore, in the case where an electron beam is used, the structure could be such that either a mask is used or a pattern can be directly formed on a substrate without the use of a mask.

The optical assembly 16 projects and/or focuses the light passing through the reticle 32 to the wafer 34. Depending upon the design of the exposure apparatus 10, the optical assembly 16 can magnify or reduce the image illuminated on the reticle 32. The optical assembly 16 need not be limited to a reduction system. It could also be a 1× or magnification system.

When far ultra-violet rays such as the excimer laser is used, glass materials such as quartz and fluorite that transmit far ultra-violet rays can be used in the optical assembly 16. When the F₂ type laser or x-ray is used, the optical assembly 16 can be either catadioptric or refractive (a reticle should also preferably be a reflective type), and when an electron beam is used, electron optics can consist of electron lenses and deflectors. The optical path for the electron beams should be in a vacuum.

Also, with an exposure device that employs vacuum ultra-violet radiation (VUV) of wavelength 200 nm or lower, use of the catadioptric type optical system can be considered. Examples of the catadioptric type of optical system include the disclosure Japan Patent Application Disclosure No. 8-171054 published in the Official Gazette for Laid-Open Patent Applications and its counterpart U.S. Pat. No, 5,668,672, as well as Japan Patent Application Disclosure No. 10-20195 and its counterpart U.S. Pat. No. 5,835,275. In these cases, the reflecting optical device can be a catadioptric optical system incorporating a beam splitter and concave mirror. Japan Patent Application Disclosure No. 8-334695 published in the Official Gazette for Laid-Open Patent Applications and its counterpart U.S. Pat. No. 5,689,377 as well as Japan Patent Application Disclosure No. 10-3039 and its counterpart U.S. patent application Ser. No. 873,605 (Application Date: Jun. 12, 1997) also use a reflecting-refracting type of optical system incorporating a concave mirror, etc., but without a beam splitter, and can also be employed with this invention. As far as is permitted, the disclosures in the above-mentioned U.S. patents, as well as the Japan patent applications published in the Official Gazette for Laid-Open Patent Applications are incorporated herein by reference.

The reticle stage assembly 18 holds and positions the reticle 32 relative to the optical assembly 16 and the wafer 34. Somewhat similarly, the wafer stage assembly 20 holds and positions the wafer 34 with respect to the projected image of the illuminated portions of the reticle 32.

Further, in photolithography systems, when linear motors (see U.S. Pat. Nos. 5,623,853 or 5,528,118) are used in a wafer stage or a mask stage, the linear motors can be either an air levitation type employing air bearings or a magnetic levitation type using Lorentz force or reactance force. Additionally, the stage could move along a guide, or it could be a guideless type stage that uses no guide. As far as is permitted, the disclosures in U.S. Pat. Nos. 5,623,853 and 5,528,118 are incorporated herein by reference.

Alternatively, one of the stages could be driven by a planar motor, which drives the stage by an electromagnetic force generated by a magnet unit having two-dimensionally arranged magnets and an armature coil unit having two-dimensionally arranged coils in facing positions. With this type of driving system, either the magnet unit or the armature coil unit is connected to the stage and the other unit is mounted on the moving plane side of the stage.

Movement of the stages as described above generates reaction forces that can affect performance of the photolithography system. Reaction forces generated by the wafer (substrate) stage motion can be mechanically transferred to the floor (ground) by use of a frame member as described in U.S. Pat. No. 5,528,100 and published Japanese Patent Application Disclosure No. 8-136475. Additionally, reaction forces generated by the reticle (mask) stage motion can be mechanically transferred to the floor (ground) by use of a frame member as described in U.S. Pat. No. 5,874,820 and published Japanese Patent Application Disclosure No. 8-330224. As far as is permitted, the disclosures in U.S. Pat. Nos. 5,528,100 and 5,874,820 and Japanese Patent Application Disclosure No. 8-330224 are incorporated herein by reference.

The measurement system 22 monitors movement of the reticle 32 and the wafer 34 relative to the optical assembly 16 or some other reference. With this information, the control system 24 can control the reticle stage assembly 18 to precisely position the reticle 32 and the wafer stage assembly 20 to precisely position the wafer 34. For example, the measurement system 22 can utilize multiple laser interferometers, encoders, and/or other measuring devices.

The control system 24 is connected to the reticle stage assembly 18, the wafer stage assembly 20, and the measurement system 22 (the stage mover assembly 26). The control system 24 receives information from the measurement system 22 and controls the stage mover assemblies 18, 20 to precisely position the reticle 32 and the wafer 34. The control system 24 can include one or more processors and circuits.

A photolithography system (an exposure apparatus) according to the embodiments described herein can be built by assembling various subsystems, including each element listed in the appended claims, in such a manner that prescribed mechanical accuracy, electrical accuracy, and optical accuracy are maintained. In order to maintain the various accuracies, prior to and following assembly, every optical system is adjusted to achieve its optical accuracy. Similarly, every mechanical system and every electrical system are adjusted to achieve their respective mechanical and electrical accuracies. The process of assembling each subsystem into a photolithography system includes mechanical interfaces, electrical circuit wiring connections and air pressure plumbing connections between each subsystem. Needless to say, there is also a process where each subsystem is assembled prior to assembling a photolithography system from the various subsystems. Once a photolithography system is assembled using the various subsystems, a total adjustment is performed to make sure that accuracy is maintained in the complete photolithography system. Additionally, it is desirable to manufacture an exposure system in a clean room where the temperature and cleanliness are controlled.

The present invention can be utilized in an immersion type exposure apparatus with taking suitable measures for a liquid. For example, PCT Patent Application WO 99/49504 discloses an exposure apparatus in which a liquid is supplied to the space between a substrate (wafer) and a projection lens system in exposure process. As far as is permitted, the disclosures in WO 99/49504 are incorporated herein by reference.

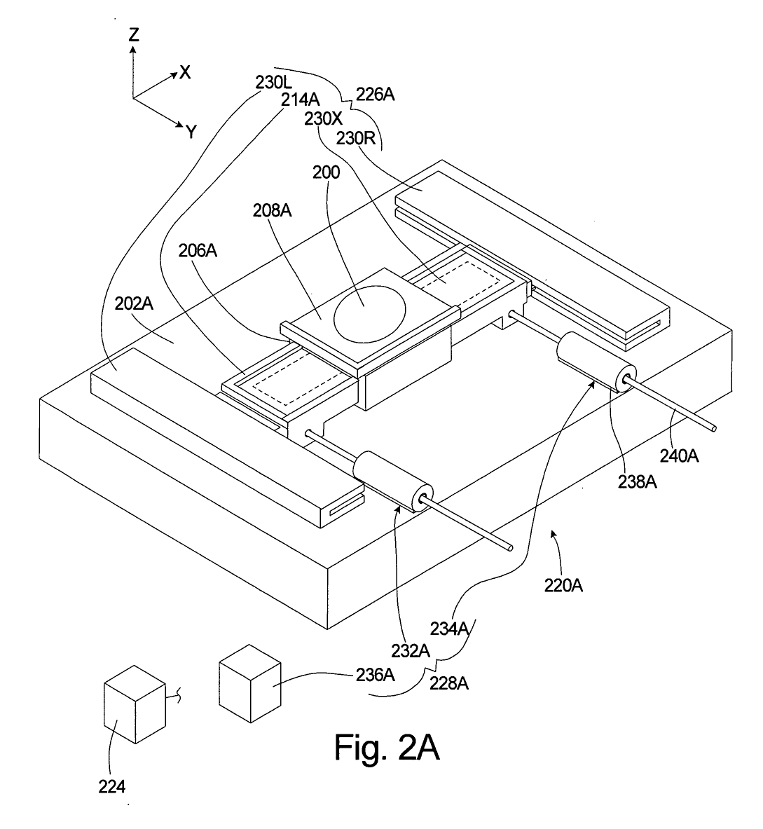

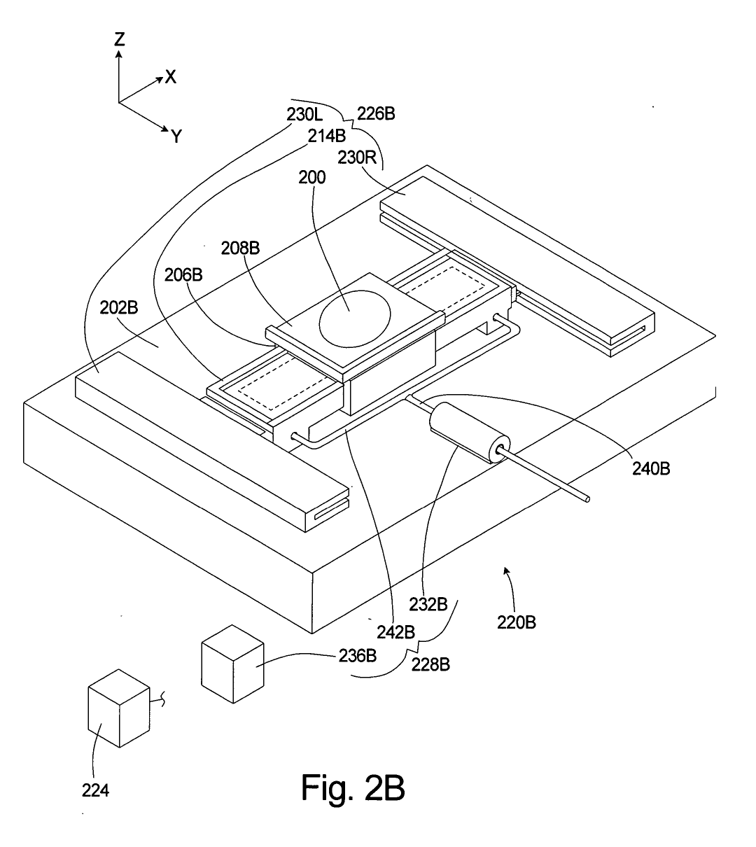

FIG. 2A is a perspective view of a control system 224 and a first embodiment of a stage assembly 220A that is used to position a device 200. For example, the stage assembly 220A can be used as the wafer stage assembly 20 in the exposure apparatus 10 of FIG. 1. In this embodiment, the stage assembly 220A positions the wafer 34 (illustrated in FIG. 1) during manufacturing of the semiconductor wafer 34. Alternatively, the stage assembly 220A can be used to move other types of devices 200 during manufacturing and/or inspection, to move a device under an electron microscope (not shown), or to move a device during a precision measurement operation (not shown). For example, the stage assembly 220A could be designed to function as the reticle stage assembly 18.

The stage assembly 220A includes a stage base 202A, a stage mover assembly 226A, a stage 206A, and a device table 208A. The design of the components of the stage assembly 220A can be varied. For example, in FIG. 2A, the stage assembly 220A includes one stage 206A. Alternatively, however, the stage assembly 220A could be designed to include more than one stage 206A.

In FIG. 2A, the stage base 202A is generally rectangular shaped. Alternatively, the stage base 202A can be another shape. The stage base 202A supports some of the components of the stage assembly 220A above the mounting base 36.

The stage mover assembly 226A controls and moves the stage 206A and the device table 208A relative to the stage base 202A. For example, the stage mover assembly 226A can move the stage 206A with three degrees of freedom, less than three degrees of freedom, or six degrees of freedom relative to the stage base 202A. The stage mover assembly 226A can include one or more movers, such as rotary motors, voice coil motors, linear motors utilizing a Lorentz force to generate drive force, electromagnetic movers, planar motor, or some other force movers.

In FIG. 2A, the stage mover assembly 226A includes a left Y mover 230L, a right Y mover 230R, a guide bar 214A, an X mover 230X (illustrated in phantom), and a force provider assembly 228A.

The Y movers 230L, 230R move the guide bar 214A, the stage 206A and the device table 208A with a relatively large displacement along the Y axis and with a limited range of motion about the Z axis, and the X mover 230X moves the stage 206A and the device table 208A with a relatively large displacement along the X axis relative to the guide bar 214A.

The design of each mover 230L, 230R, 230X can be varied to suit the movement requirements of the stage mover assembly 226A. In the embodiment illustrated in FIG. 2A, each of the movers 230L, 230R, 230X is a linear motor.

The guide bar 214A guides the movement of the stage 206A along the X axis. In FIG. 2A, the guide bar 214A is somewhat rectangular beam shaped. A bearing (not shown) maintains the guide bar 214A spaced apart along the Z axis relative to the stage base 202A and allows for motion of the guide bar 214A along the Y axis and about the Z axis relative to the stage base 202A.

In FIG. 2A, the stage 206A moves with the guide bar 214A along the Y axis and about the Z axis and the stage 206A moves along the X axis relative to the guide bar 214A. In this embodiment, the stage 206A is generally rectangular shaped and includes a rectangular shaped opening for receiving the guide bar 214A. A bearing (not shown) maintains the stage 206A spaced apart along the Z axis relative to the stage base 202A and allows for motion of the stage 206A along the X axis, along the Y axis and about the Z axis relative to the stage base 202A.

Further, the stage 206A is maintained apart from the guide bar 214A with opposed bearings (not shown) that allow for motion of the stage 206A along the X axis relative to the guide bar 214A, while inhibiting motion of the stage 206A relative to the guide bar 214A along the Y axis and about the Z axis.

In the embodiment illustrated in the FIG. 2A, the device table 208A is generally rectangular plate shaped and includes a clamp that retains the device 200. Further, the device table 208A is fixedly secured to the stage 206A and moves concurrently with the stage 206A. Alternatively, for example, the stage mover assembly 226A can include a table mover assembly (not shown) that moves and adjusts the position of the device table 208A relative to the stage 206A. For example, the table mover assembly can adjust the position of the device table 208A relative to the stage 206A with six degrees of freedom. Alternatively, for example, the table mover assembly can move the device table 208A relative to the stage 206A with only three degrees of freedom.

In one embodiment, the force provider assembly 228A is useful with a stage assembly 220A that repetitively moves that stage 206A along one axis, such as the Y axis. For example, in FIG. 2A, the force provider assembly 228A is used in conjunction with the Y movers 230L, 230R to increase the peak force achievable by the Y movers 230L, 230R alone along the Y axis, while being able to provide accuracy control. In this embodiment, the force provider assembly 228A is used in parallel with other Y movers 230L, 230R. In one embodiment, the control system 224 actively controls the Y movers 230L, 230R to precisely position the stage 206A along the Y axis. In this embodiment, the force provider assembly 228A is not actively controlled and the force provider assembly 228A is used to increase the peak force achievable by the stage mover assembly 226A along the Y axis.

The design of the force provider assembly 228A can vary. For example, in FIG. 2A, the force provider assembly 228A includes a first force provider 232A, a second force provider 234A, and a fluid source 236A.

In one embodiment, each force provider 232A, 234A is a pneumatic type cylinder that includes a provider housing 238A and a piston assembly 240A. In FIG. 2A, the provider housing 238A of each force provider 232A, 234A is fixedly secured to stage base 202A. Alternatively, for example, the provider housing 238A of one or both of the force providers 232A, 234A can be secured with resilient members (not shown) to the stage base 202A, the provider housing 238A of one or both of the force providers 232A, 234A is secured to a reaction frame (not shown) instead of the stage base 202A, or the provider housing 238A of one or both of the force providers 232A, 234A is secured to a reaction mass (not shown).

Further, in FIG. 2A, the piston assembly 240A is secured and coupled to the load, e.g. the stage 206A via the guide bar 214A. More specifically, the piston assembly 240A of the first force provider 232A is secured to the guide bar 214A near the left Y mover 230L and the piston assembly 240A of the second force provider 234A is secured to the guide bar 214A near the right Y mover 230R. With this design, the first force provider 232A, is connected in parallel with the left Y mover 230L and the second force provider 234A is connected in parallel with the right Y mover 230R.

Alternatively, for example, the force provider assembly 228A could be designed to include an X force provider (not shown) that is coupled to the stage 206A to act in parallel with the X mover 230X and increase the peak force achievable along the X axis.

Each bearing, for example, can be a vacuum preload type fluid bearing, a magnetic type bearing or a roller type assembly.

Further, this invention can be utilized in an exposure apparatus that comprises two or more substrate and/or reticle stages. In such apparatus, the additional stage may be used in parallel or preparatory steps while the other stage is being used for exposing. Such a multiple stage exposure apparatus are described, for example, in Japan Patent Application Disclosure No. 10-163099 as well as Japan Patent Application Disclosure No. 10-214783 and its counterparts U.S. Pat. No. 6,341,007, No. 6,400,441, No. 6,549,269, and No. 6,590,634. Also it is described in Japan Patent Application Disclosure No. 2000-505958 and its counterparts U.S. Pat. No. 5,969,411 as well as U.S. Pat. No. 6,208,407. As far as is permitted, the disclosures in the above-mentioned U.S. Patents, as well as the Japan Patent Applications, are incorporated herein by reference.

This invention can be utilized in an exposure apparatus that has a movable stage retaining a substrate (wafer) for exposing it, and a stage having various sensors or measurement tools for measuring, as described in Japan Patent Application Disclosure 11-135400. As far as is permitted, the disclosures in the above-mentioned Japan patent application are incorporated herein by reference.

FIG. 2B is a perspective view of another embodiment of a stage assembly 220B and a control system 224 that is used to position the device 200. For example, the stage assembly 220B can be used as the wafer stage assembly 20 or the reticle stage assembly 18 in the exposure apparatus 10 of FIG. 1. Alternatively, the stage assembly 220B can be used to move other types of devices 200.

The stage assembly 220B includes a stage base 202B, a stage mover assembly 226B, a stage 206B, and a device table 208B that are somewhat similar to the corresponding components described above. However, in this embodiment, the stage mover assembly 226B includes a force provider assembly 228B that is somewhat different.

More specifically, in FIG. 2B, the force provider assembly 228B includes a force provider 232B, a fluid source 236B and a provider connector 242B that couples and secures a piston assembly 240B of the force provider 232B to the guide bar 214B. In this embodiment, the provider connector 242B connects the piston assembly 240B of the force provider 232B to the guide bar 214B near the left Y mover 230L and the right Y mover 230R. With this design, the force provider 232B is connected in parallel with the left Y mover 230L and the right Y mover 230R. In one embodiment, the provider connector 242B is a beam that extends between the ends of the guide bar 214B and allows the stage 206B to move relative to the guide bar 214B and the provider connector 242B.

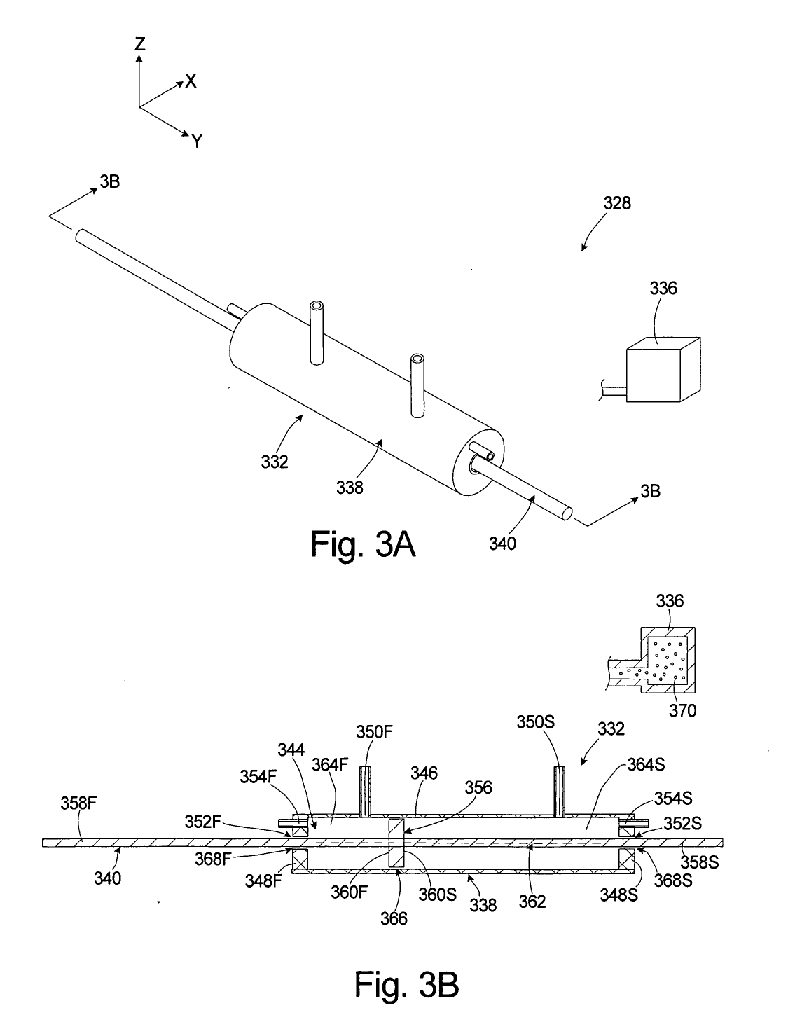

FIGS. 3A is a perspective view of a force provider assembly 328 that can be used in the stage assembly 220A, 220B illustrated in FIG. 2A, FIG. 2B or another type of stage assembly. Alternatively, the force provider assembly 328 can be used in another type of system to move or position another type of device or object during a manufacturing, measurement and/or inspection process.

The design of the force provider assembly 328 can be varied to suit the design requirements of the system. In FIG. 3A, the force provider assembly 328 includes a force provider 332 and a fluid source 336. Alternatively, for example, the force provider assembly can be designed without the fluid source or with multiple force providers. In this embodiment, the force provider 332 is a pneumatic type actuator that includes a provider housing 338 and a piston assembly 340.

FIG. 3B is a cross-sectional view of the force provider 332 taken on line 3B-3B and a cross-sectional view of the fluid source 336 of FIG. 3A. In this embodiment, the provider housing 338 defines a piston chamber 344 and includes a tubular, cylinder wall 346, a disk shaped first side wall 348F positioned at a first end of the cylinder wall 346, and a disk shaped second side wall 348S positioned at a second end of the cylinder wall 346. The size and shape of the cylinder wall 346 can be varied to suit the design and force requirements of the force provider 332. In this embodiment, the cylinder wall 346 is generally annular shaped. Alternatively, for example, the cylinder wall 346 could be square tube shaped. The cylinder wall 346 includes a first cylinder aperture 350F and a spaced apart, second cylinder aperture 350S that extend transversely through the cylinder wall 346. In FIG. 3B, each side wall 348F, 348S is generally annular disk shaped. The first side wall 348F includes a first beam aperture 352F for receiving a portion of the piston assembly 340 and a first fluid inlet 354F that is in fluid communication with the fluid source 336. Similarly, the second side wall 348S includes a second beam aperture 352S for receiving a portion of the piston assembly 340 and a second fluid inlet 354S that is in fluid communication with the fluid source 336. Alternatively, for example, the fluid inlets 354F, 354S could be at another location, such as through the cylinder wall 346 near each end.

In one embodiment, the cylinder apertures 350F, 350S are open and exposed to atmospheric pressure or the room pressure that surrounds the force provider 332. With this design, for example, the cylinder apertures 350F, 350S are each in fluid communication with a fluid that is at a first pressure. In FIG. 3B, the first pressure is atmospheric pressure, approximately 14.7 PSI. Stated another way, the pressure in the first cylinder aperture 350F is approximately equal to the pressure in the second cylinder aperture 350S. For example, in alternative embodiments, the pressure difference between the cylinder apertures 350F, 350S is approximately 0, 0.1, 0.5, 1, 2, or 3 PSI.

The piston assembly 340 includes a piston 356, a rigid first beam 358F and a rigid second beam 358S. In this embodiment, the piston 356 is somewhat flat disk shaped, has a generally circular shaped cross section, and includes a first piston side 360F and a second piston side 360S. The piston 356 is sized and shaped to fit within the piston chamber 344 and move relative to the provider housing 338 along a piston path 362 (illustrated with a dashed line).

The first beam 358F is generally rod shaped, includes a proximal end that is secured to the first piston side 360F and a distal end that is positioned outside the provider housing 338. Stated another way, the first beam 358F cantilevers away from the piston 356 and extends through the first beam aperture 352F. Similarly, the second beam 358S is generally rod shaped, includes a proximal end that is secured to the second piston side 360S and a distal end that is positioned outside the provider housing 338. The second beam 358S cantilevers away from the piston 356 and extends through the second beam aperture 352S.

In one embodiment, the distal end of one of the beams 358F, 358S is coupled and secured to the load, e.g. the guide bar 214A (illustrated in FIG. 2A).

In FIG. 3B, when the piston 356 is to the left of the first cylinder aperture 350F the piston 356 cooperates with the cylinder wall 346 and the first side wall 348F to define a first chamber 364F on the first piston side 360F, and when the piston is to the right of the second cylinder aperture 350S the piston 356 cooperates with the cylinder wall 346 and the second side wall 348S to define a second chamber 364S on the second piston side 360S.

In one embodiment, a wall gap 366 exists between the piston 356 and the cylinder wall 346, a first beam gap 368F exists between the first beam 358F and the first side wall 348F, and a second beam gap 368S exists between the second beam 358S and the second side wall 348S. It should be noted that the gaps 366, 368F, 368S are greatly exaggerated herein. With this design, the piston assembly 340 moves freely and with little friction relative to the provider housing 338. In one embodiment, the piston assembly 340 is supported by a mechanical bearing or an air bearing.

Alternatively, a piston seal (not shown) can be used in the wall gap 366, a first seal (not shown) can be used in the first beam gap 368F and/or a second seal (not shown) can be used in the second beam gap 368S. In one embodiment, each seal is a low friction type seal that allows for easy motion of the piston assembly 340 relative to the provider housing 338.

The fluid source 336 is in fluid communication with the fluid inlets 354F, 354S. For example, the fluid source 336 can be connected with conduits to the fluid inlets 354F, 354S. With this design, the fluid source 336 can selectively direct pressurized fluid 370 (illustrated as circles) to the fluid inlets 354F, 354S, respectively and into the chambers 364F, 364S, respectively. The fluid source 336 can be controlled by the control system 224 (illustrated in FIG. 2A). In one embodiment, the fluid source 336 is a fluid pump. Alternatively, the fluid source 336 can be a container of pressurized fluid. It should be noted that the fluid source 336 can include multiple fluid sources. In the embodiments provided herein, the fluid source 336 can be controlled by passive pressure regulation or an active pneumatic servo valve. In the case of active controlling, feedback and feed forward control can be applied to the serving the pneumatic valve to optimize pneumatic force performance.

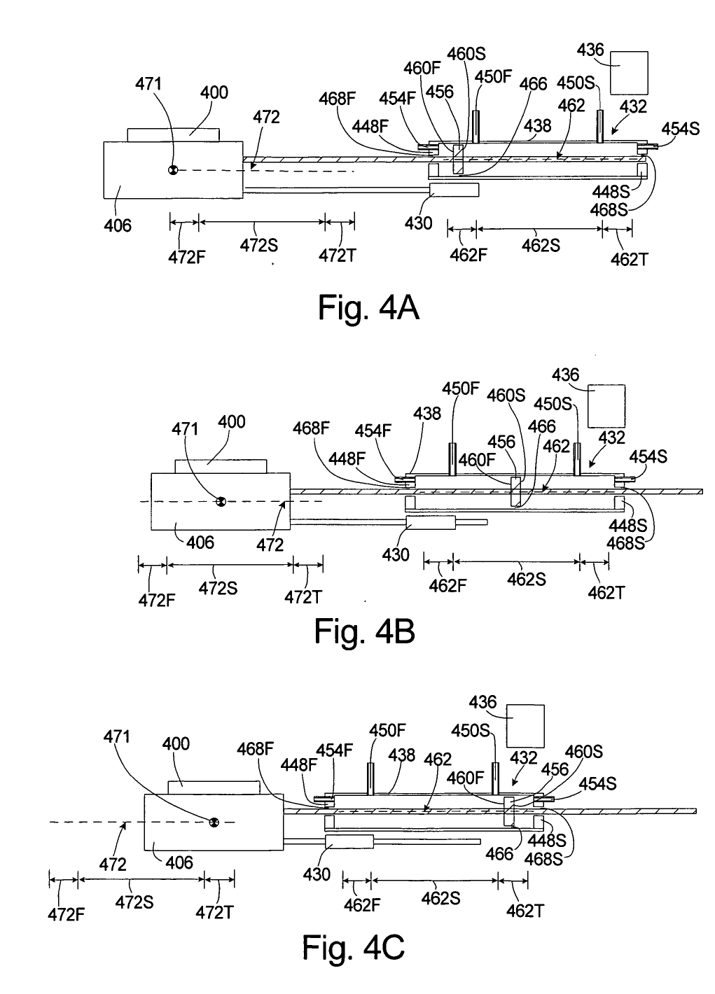

FIGS. 4A-4C each illustrate a cross-sectional view of a force provider 432 and a simplified illustration of a mover 430 coupled to a stage 406, a fluid source 436, and a device 400. FIGS. 4A-4C illustrate movement of a center of gravity 471 (c.g.) of the stage 406 by the mover 430 and the force provider 432 along a stage path 472 that includes a first stage region 472F, a second stage region 472S, and a third stage region 472T. In FIG. 4A, the c.g. 471 of the stage 406 is in the first stage region 472F; in FIG. 4B, the c.g. 471 of the stage 406 is in the second stage region 472S; and in FIG. 4C, the c.g. 471 of the stage 406 is in the third stage region 472T.

In one embodiment, in the first stage region 472F and the third stage region 472T, the mover 430 and the force provider 432 provide an acceleration/deceleration force on the stage 406 that accelerates and decelerates the stage 406, and in the second stage region 472S, the mover 430 moves the stage 406 at a constant velocity. In this embodiment, the first stage region 472F and the third stage region 472T are also referred to as acceleration/deceleration regions, and the second stage region 472S is also referred to a constant velocity region. In one embodiment, processing of the device 400 occurs while the stage 406 and the device 400 are moved at constant velocity in the second stage region 472S.

It should be noted that the control system 224 (illustrated in FIG. 2A) controls the mover 430 to precisely position and move the stage 406 back and forth along the stage path 472. One movement of the stage 406 along the stage path 472 is described below. Starting with the stage 406 in the constant velocity region 472S (illustrated in FIG. 4B) moving right to left along the stage path 472, at this time the mover 430 controls the trajectory of the stage 406 so that the stage 406 is moved at constant velocity. Once the c.g. 471 of the stage 406 enters the first stage region 472F (illustrated in FIG. 4A), the mover 430 and the force provider 432 act in parallel to decelerate the stage 406. When the stage 406 is at the left end of the stage path 472, the stage 406 will be stopped by the mover 430. Subsequently, the stage 406 is accelerated from left to right along the stage path 472 by the mover 430 and the force provider 432. When the c.g. 471 of the stage 406 enters the constant velocity region 472S (illustrated in FIG. 4B) moving left to right, at this time the mover 430 controls the trajectory of the stage 406 so that the stage 406 is moved at constant velocity. Once the c.g. 471 of the stage 406 enters the third stage region 472T (illustrated in FIG. 4C), the mover 430 and the force provider 432 act in parallel to decelerate the stage 406. When the stage 406 is at the right end of the stage path 472, the stage 406 will be stopped by the mover 430. Subsequently, the stage 406 is accelerated from right to left along the stage path 472 by the mover 430 and the force provider 432. Subsequently, the c.g. 471 of the stage 406 enters the constant velocity region 472S (illustrated in FIG. 4B) moving right to left. In this embodiment, the mover 430 always controls the trajectory of the stage 406 so that the stage 406 follows the desired trajectory. This procedure can be repeated for motion of the stage 406 along the Y axis.

FIGS. 4A-4C also illustrate the operation of the force provider 432 during this time. In one embodiment, the piston 456 moves relative to the provider housing 438 along a piston path 462 that includes a first piston region 462F, a second piston region 462S, and a third piston region 462T. In FIG. 4A, the piston 456 is in the first piston region 462F; in FIG. 4B, the piston 456 is in the second piston region 462S; and in FIG. 4C, the piston 456 is in the third piston region 462T. In this embodiment, (i) the piston 456 is in the first piston region 462F when the c.g. 471 of the stage 406 is in the first stage region 472F; (ii) the piston 456 is in the second piston region 462S when the c.g. 471 of the stage 406 is in the second stage region 472S; and (iii) the piston 456 is in the third piston region 462T when the c.g. 471 of the stage 406 is in the third stage region 472T. In this embodiment, the size of the regions 462F-462T can be changed by changing the location of the cylinder apertures 450F, 450S.

In the first piston region 462F, the piston 456 is positioned between the first side wall 448F and the first cylinder aperture 450F. In the second piston region 462S, the piston 456 is positioned between the first cylinder aperture 450F and second cylinder aperture 450S. In the third piston region 462T, the piston 456 is positioned between the second cylinder aperture 450S and the second side wall 448S.

In one embodiment, when the piston 456 is in the first piston region 462F and in the third piston region 462T, the force provider 432 provides an acceleration/deceleration force on the stage 406, and in the second piston region 462S, the force provider 432 exerts substantially no force on the stage 406 and the stage 406 moves at a constant velocity. In this embodiment, the first piston region 462F and the third piston region 462T are also referred to as acceleration/deceleration regions, and the second piston region 462S is also referred to a constant velocity region.

One back and forth movement of the piston 456 along the piston path 462 is described below. It should be noted that at all times, the mover 430 controls the trajectory and/or position of the stage 406. Starting with the piston 456 in the constant velocity region 462S (illustrated in FIG. 4B) moving right to left along the piston path 462, the piston 456 is between the cylinder apertures 450F, 450S and the pressure on both sides of the piston 456 is approximately equal. At this time, the piston 456 is moved by the mover 430 along with the stage 406. Because the pressure is approximately equal on both sides of the piston 456 at this time, approximately no force will be acting on the piston 456. This minimizes transmissibility between the force provider 432 and the stage 406.

Once the piston 456 enters the first piston region 462F on the left of the first cylinder aperture 450F, the force provider 432 acts in parallel with the mover 430 to decelerate the stage 406. More specifically, with the piston 456 moving to the left entering the first piston region 462F, the mover 430 starts providing force to decelerate the stage 406. At the same time, the piston 456 passes the first cylinder aperture 450F and the volume of air to the left of the first cylinder aperture 450F will start compressing and the pressure on the first piston side 460F is greater than the pressure on the second piston side 460S. This creates a resultant pressure that is derived from the equation PV=nRT; where P is the absolute pressure in the closed system, V is the volume of this closed system, n is the unit of air, R is the gas constant and T is the temperature. Assuming that the air leaking through the wall gap 466 and the first beam gap 468F will be replaced by the fluid supplied by the fluid source 436 in the first fluid inlet 454F, n will be constant. In addition, also assume that temperature T is constant. Then P∝1/V. The resultant force provided by the forcer provider 432 would just be equal to the area of the piston 456 times P (gauge pressure). Since the volume V decreases as the piston 456 moves to the left, the resultant force increases, adding a deceleration force from the force provider 432 in addition to the deceleration force provided by the mover 430. As a result, the peak force achievable for deceleration will be higher than with the mover 430 alone. The force output is a function of the compressed volume. If the volume compressed to ½ of the starting volume, the force from the force provider 432 will be 1 atm pressure times the active pressure area of the piston 456.

Eventually, the stage 406 will come to a complete stop. At this point in time, the mover 406 will still be providing force in the same direction, but the stage 406 would now start to accelerate to the right along the stage path 472. Meanwhile, the positive pressure built up on the first piston side 460F will still be adding an acceleration force from the force provider 432 to the force output from the mover 430. Thus, the stage 406 is accelerated from left to right along the stage path 472 by the mover 430 and the force provider 432. When, the stage 406 enters the constant velocity region 472S (illustrated in FIG. 4B) moving left to right, at this time the mover 430 controls the trajectory of the stage 406 so that the stage 406 is moved at constant velocity. At this time, the piston 456 is in the second piston region 462S and the pressure on both sides of the piston 456 is equal. Once the stage 406 enters the third stage region 472T, the piston 456 is in the third piston region 462T, the mover 430 and the force provider 432 act in parallel to decelerate the stage 406. At this time, the piston 456 passes the second cylinder aperture 450S and the volume of air to the right of the second cylinder aperture 450S will start compressing and the pressure on the second piston side 460S is greater than the pressure on the first piston side 460F. This results in a deceleration force from the force provider 432 on the stage 406. When the stage 406 is at the right end of the stage path 472, the stage 406 will be stopped by the mover 430. Subsequently, the stage 406 is accelerated from right to left along the stage path 472 by the mover 430 and the force provider 432. When, the stage 406 enters the constant velocity region 472S (illustrated in FIG. 4B) moving right to left, at this time the mover 430 controls the trajectory of the stage 406 so that the stage 406 is moved at constant velocity. This procedure can be repeated for motion of the stage 406 along the Y axis.

In one embodiment, the force provider 432 cannot be used alone and has no capability of position control and the force provider 432 provides force in a position where the volume has been compressed. In this embodiment, the force provider 432 is not actively controlled and a gauge pressure of zero is measured at each cylinder aperture 450F, 450S when the piston 456 is in the second piston region 462S.

In one embodiment, the fluid source 436 compensates for (i) fluid lost in the wall gap 466 and the first beam gap 468F when the piston 456 is in the first piston region 462F and (ii) fluid lost in the wall gap 466 and the second beam gap 468S when the piston 456 is in the second piston region 462S. For example, in one embodiment, (i) when the piston 456 is in the first piston region 462F, the amount of fluid directed into first fluid inlet 454F by the fluid source 436 is approximately equal to the amount of fluid that escapes from the wall gap 466 and the first beam gap 468F; (ii) when the piston 456 is in the third piston region 462T, the amount of fluid directed into the second fluid inlet 454S by the fluid source 436 is approximately equal to the amount of fluid that escapes from the wall gap 466 and the second beam gap 468S; and (iii) the fluid source 436 does not direct fluid into the fluid inlets 454F, 454S when the piston 456 is in the second piston region 462S.

In one embodiment, the fluid source 436 directs fluid into the first fluid inlet 454F so that the pressure on the first piston side 460F does not decrease when the piston 456 is in the first piston region 462F and the fluid source 436 directs fluid into the second fluid inlet 454S so that the pressure on the second piston side 460S does not decrease when the piston 456 is in the third piston region 462T.

In another embodiment, the amount of fluid loss when the piston 456 is in the first piston region 462F and/or the third piston region 462T is empirically calculated and the control system controls the fluid source to compensate for the fluid loss. In alternative embodiments, the fluid source 436 directs fluid to the fluid inlets 454F, 454S at a rate of approximately 0.5, 1, 2, 3, 4 or 5 liters/minute.

In another embodiment, the force provider 432 provides dampening in addition or alternatively to an acceleration/deceleration force. This is accomplished by slowly leaking fluid when the piston 456 is in the first piston region 462F or the third piston region 462T.

It should be noted that in one embodiment, the rate in which pressure on the piston 456 increases and decreases will vary according to the volume being compressed in the first chamber 364F and the second chamber 364S. Smaller original volumes for the first chamber 364F and the second chamber 364S will result in more rapid increases and decreases of pressure against the piston 456. As a result thereof, an external reservoir (not shown) can be connected to the chambers 364F, 364S to change the volume of fluid being compressed.

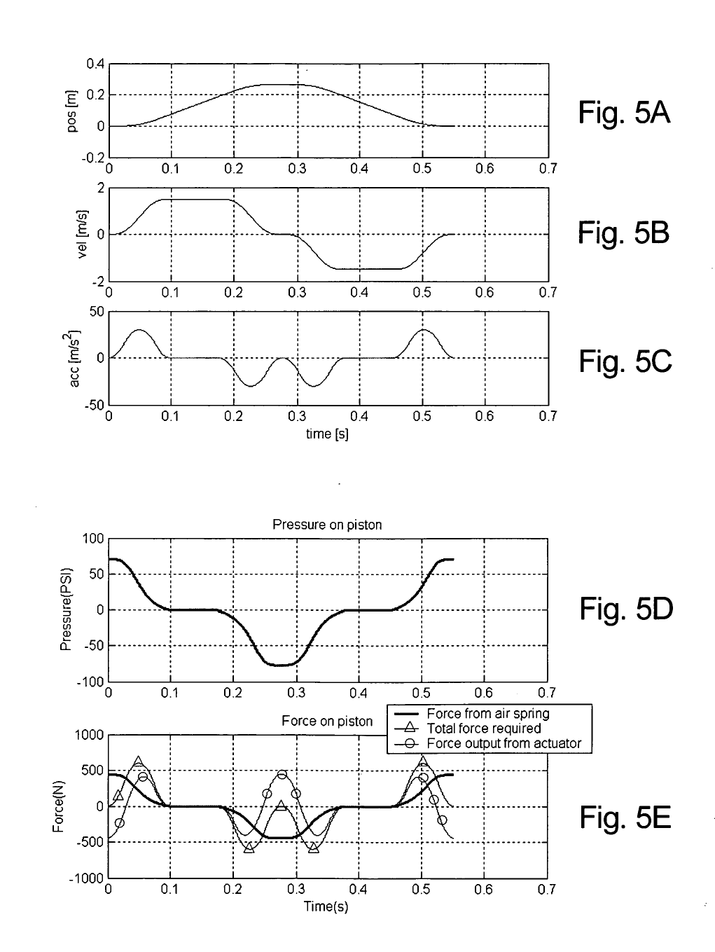

FIG. 5A is a graph that illustrates an example of the position of the stage versus time during movement of the stage along the stage path from the first stage region, through the second stage region to the third stage region and from the third stage region through the second stage region back to the first stage region.

FIG. 5B is a graph that illustrates one example of velocity of the stage versus time during movement of the stage along the stage path from the first stage region, through the second stage region to the third stage region and from the third stage region through the second stage region back to the first stage region.

FIG. 5C is a graph that illustrates one example of acceleration versus time during movement of the stage along the stage path from the first stage region, through the second stage region to the third stage region and from the third stage region through the second stage region back to the first stage region.

FIG. 5D is a graph that illustrates one example of pressure on the piston versus time during movement of the stage along the stage path from the first stage region, through the second stage region to the third stage region and from the third stage region through the second stage region back to the first stage region.

FIG. 5E is a graph that illustrates one example of force from the piston (air spring), force from mover (actuator) and total force required on the stage versus time during movement of the stage along the stage path from the first stage region, through the second stage region to the third stage region and from the third stage region through the second stage region back to the first stage region.

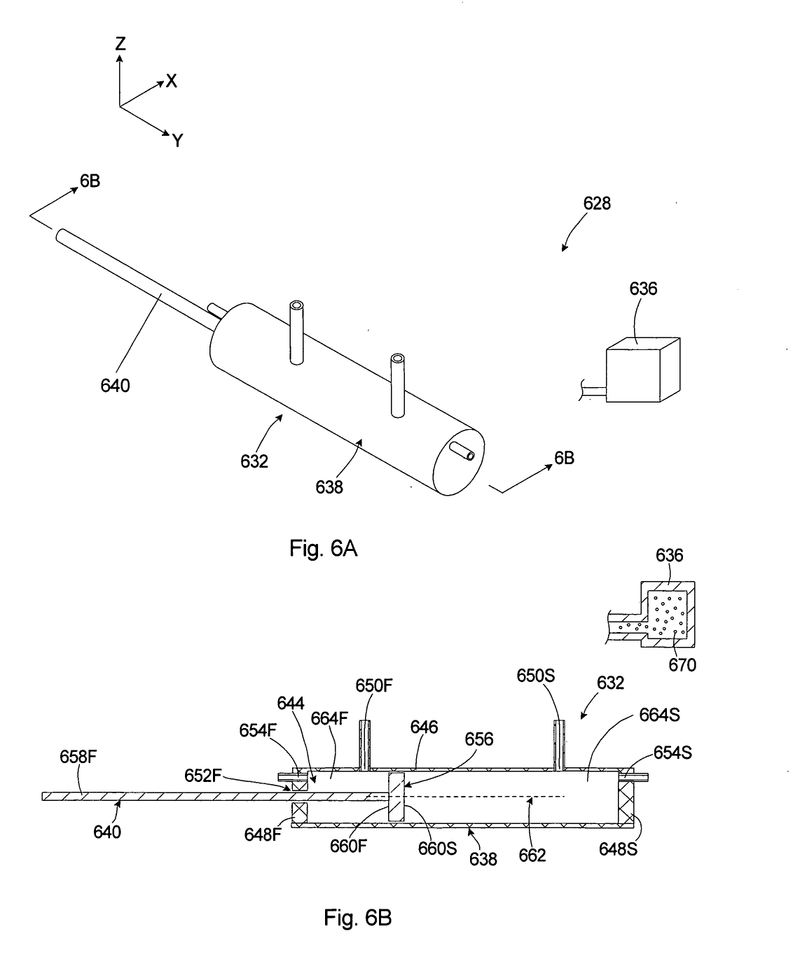

FIGS. 6A is a perspective view of another embodiment of a force provider assembly 628 that can be used in the stage assembly 220A, 220B illustrated in FIG. 2A, FIG. 2B or another type of stage assembly. Alternatively, the force provider assembly 628 can be used in another type of system to move or position another type of device or object during a manufacturing, measurement and/or inspection process.

In FIG. 6A, the force provider assembly 628 includes a force provider 632 and a fluid source 636 and somewhat similar to the force provider 332 described above. Alternatively, for example, the force provider assembly can be designed without the fluid source or with multiple force providers. In this embodiment, the force provider 632 includes a provider housing 638 and a piston assembly 640.

FIG. 6B is a cross-sectional view of the force provider 632 and a cross-sectional view of the fluid source 636 of FIG. 6A. In this embodiment, the provider housing 638 defines a piston chamber 644 and includes a tubular, cylinder wall 646, a first side wall 648F positioned at a first end of the cylinder wall 646, and a second side wall 648S positioned at a second end of the cylinder wall 646.

The cylinder wall 646 includes a first cylinder aperture 650F and a spaced apart, second cylinder aperture 650S that extend transversely through the cylinder wall 646. In FIG. 6B, the first side wall 648F is generally annular disk shaped and the second side wall 648S is disk shaped. The first side wall 648F includes a first beam aperture 652F for receiving a portion of the piston assembly 640 and a first fluid inlet 654F that is in fluid communication with the fluid source 636. The second side wall 648S includes a second fluid inlet 654S that is in fluid communication with the fluid source 636. Alternatively, for example, the fluid inlets 654F, 654S could be at another location.

The piston assembly 640 includes a piston 656, and a rigid first beam 658F that are similar in design to the corresponding components described above. In FIG. 6B, when the piston 656 is to the left of the first cylinder aperture 650F, the piston 656 cooperates with the cylinder wall 646 and the first side wall 648F to define a first chamber 664F on the first piston side 660F, and when the piston 656 is to the right of the second cylinder aperture 650S, the piston 656 cooperates with the cylinder wall 646 and the second side wall 648S to define a second chamber 664S on the second piston side 660S.

The fluid source 636 is in fluid communication with the fluid inlets 654F, 654S. With this design, the fluid source 636 can selectively direct pressurized fluid 670 (illustrated as circles) to the fluid inlets 654F, 654S, respectively and into the chambers 664F, 664S, respectively and regulate the pressure in the chambers 664.

In this embodiment, the force provider 632 functions somewhat similar and provides an acceleration/deceleration force on the load (not shown in FIG. 6B) similar to the force provider 332 described above.

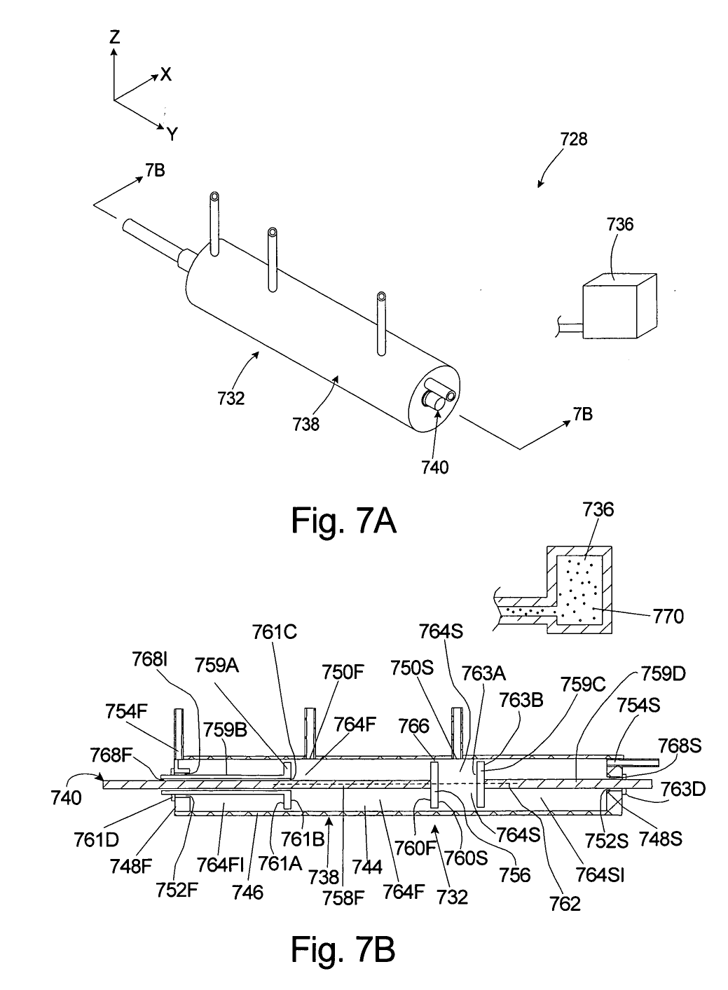

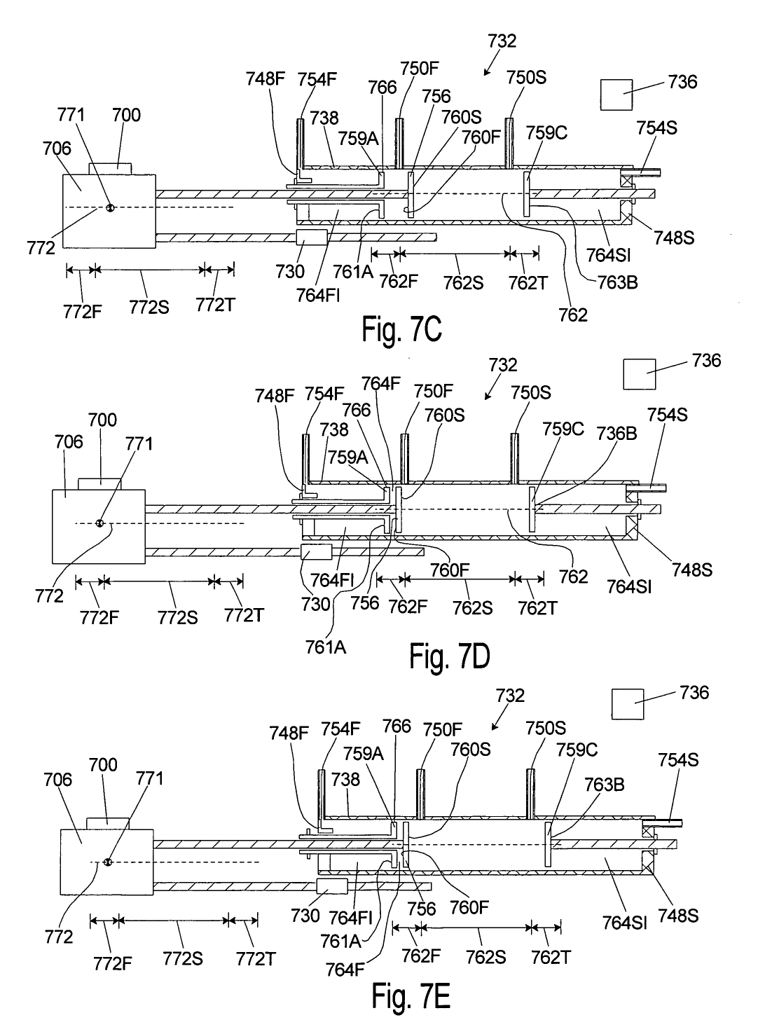

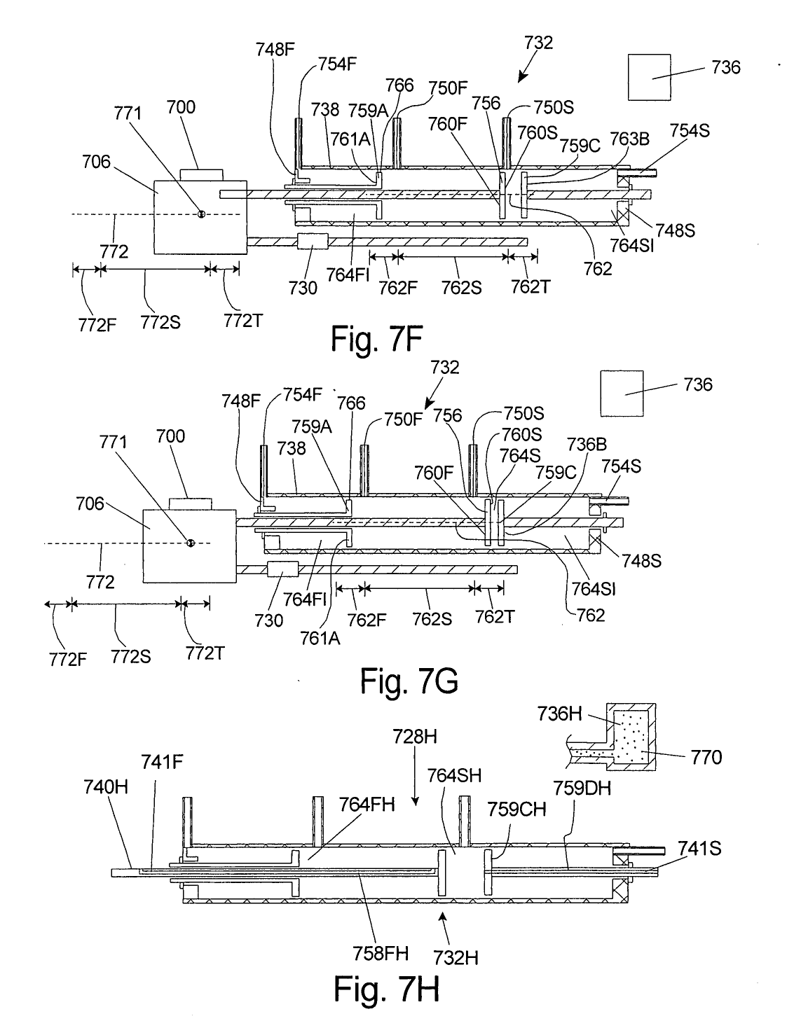

FIG. 7A is a perspective view of still another embodiment of a force provider assembly 728 that can be used in the stage assembly 220A, 220B illustrated in FIG. 2A, FIG. 2B or another type of stage assembly. Alternatively, the force provider assembly 728 can be used in another type of system to move or position another type of device or object during a manufacturing, measurement and/or inspection process.

In FIG. 7A, the force provider assembly 728 includes a force provider 732 and a fluid source 736. Alternatively, for example, the force provider assembly can be designed without the fluid source or with multiple force providers. In this embodiment, the force provider 732 includes a provider housing 738 and a piston assembly 740.

FIG. 7B is a cross-sectional view of the force provider 732 and the fluid source 736 of FIG. 7A. In this embodiment, the provider housing 738 defines a piston chamber 744 and includes a tubular, cylinder wall 746, a disk shaped first side wall 748F positioned at a first end of the cylinder wall 746, and a disk shaped second side wall 748S positioned at a second end of the cylinder wall 746.

In this embodiment, the cylinder wall 746 is generally annular shaped. The cylinder wall 746 includes a first cylinder aperture 750F, a spaced apart, second cylinder aperture 750S and a first fluid inlet 754F that extend transversely through the cylinder wall 746. The first fluid inlet 754F is in fluid communication with the fluid source 736. In FIG. 7B, each side wall 748F, 748S is generally annular disk shaped. The first side wall 748F includes a first beam aperture 752F for receiving a portion of the piston assembly 740. The second side wall 748S includes a second beam aperture 752S for receiving a portion of the piston assembly 740 and a second fluid inlet 754S that is in fluid communication with the fluid source 736. Alternatively, for example, the fluid inlets 754F, 754S could be at another location.

In one embodiment, the cylinder apertures 750F, 750S are open and exposed to atmospheric pressure or the room pressure that surrounds the force provider 732.

With this design, for example, the cylinder apertures 750F, 750S are each in fluid communication with a fluid that is at a first pressure. In FIG. 7B, the first pressure is atmospheric pressure, approximately 14.7 PSI. Stated another way, the pressure in the first cylinder aperture 750F is approximately equal to the pressure in the second cylinder aperture 750S. For example, in alternative embodiments, the pressure difference between the cylinder apertures 750F, 750S is approximately 0, 0.1, 0.5, 1, 2, or 3 PSI.

The piston assembly 740 includes a piston 756, a rigid first beam 758F, a first intermediate piston 759A, a rigid first intermediate beam 759B, a second intermediate piston 759C, and a second intermediate beam 759D. In this embodiment, the pistons 756, 759A, 759C are not fixedly secured together.

In this embodiment, the piston 756 again includes a first piston side 760F and a second piston side 760S and is sized and shaped to fit within the piston chamber 744 and move relative to the provider housing 738 along a piston path 762 (illustrated with a dashed line).

The first beam 758F is generally rod shaped, includes a proximal end that is secured to the first piston side 760F and a distal end that is positioned outside the provider housing 738. The distal end can be secured to the load, e.g. the stage (not shown in FIG. 7B). The first beam 758F cantilevers away from the piston 756 and extends through the first intermediate piston 759A, the first intermediate beam 759B, and the first beam aperture 752F.

In this embodiment, the first intermediate piston 759A is annular disk shaped and includes a first side 761A, an opposed second side 761B and a piston bar aperture 761C that is sized to receive the first beam 758F. The first intermediate piston 759A is sized and shaped to fit within the piston chamber 744 and move relative to the provider housing 738 along a portion of the piston path 762.

The first intermediate beam 759B is generally tubular shaped, includes a proximal end that is secured to the first side 761A of the first intermediate piston 759A and a distal end that is positioned outside the provider housing 738. The first intermediate beam 759B cantilevers away from the first intermediate piston 759A and extends through the first beam aperture 752F. The first intermediate beam 759B includes an aperture that receives the first beam 758F. A first stop 761D can be secured to the first intermediate beam 759B that engages the first side wall 748F and inhibits farther motion of the first intermediate beam 759B along the Y axis. The position of the first stop 761D relative to the first intermediate beam 759B can be adjusted to change the characteristics of the force provider 732.

In this embodiment, the second intermediate piston 759C is disk shaped and includes a first side 763A, an opposed second side 763B. The first intermediate piston 759C is sized and shaped to fit within the piston chamber 744 and move relative to the provider housing 738 along a portion of the piston path 762.

The second intermediate beam 759D is generally rod shaped, includes a proximal end that is secured to the second side 763B of the second intermediate piston 759C and a distal end that is positioned outside the provider housing 738. The second intermediate beam 759D cantilevers away from the second intermediate piston 759C and extends through the second beam aperture 752S. A second stop 763D can be secured to the second intermediate beam 759D that engages the second side wall 748S and inhibits farther motion of the second intermediate beam 759D along the Y axis. The position of the second stop 763D relative to the second intermediate beam 759D can be adjusted to change the characteristics of the force provider 732.

In FIG. 7B, (i) when the piston 756 is left of the first cylinder aperture 750F, the piston 756 cooperates with the cylinder wall 746 and the first intermediate piston 759A to define a first chamber 764F on first piston side 760F, (ii) when the piston is right of the second cylinder aperture 750S, the piston 756 cooperates with the cylinder wall 746 and the second intermediate piston 759C to define a second chamber 764S on second piston side 760S, (iii) the first intermediate piston 759A cooperates with the cylinder wall 746 and the first side wall 748F to define a first intermediate chamber 764FI, and (iv) the second intermediate piston 759C cooperates with the cylinder wall 746 and the second side wall 748S to define a second intermediate chamber 764SI.

In this embodiment, (i) a wall gap 766 exists between the pistons 756, 759A, 759C and the cylinder wall 746, (ii) a first beam gap 768F exists between the first beam 758F and the first intermediate beam 759B, (iii) an intermediate beam gap 7681 exists between the first intermediate beam 759B and the first side wall 748F, and (iv) a second beam gap 768S exists between the second intermediate beam 759D and the second side wall 748S. With this design, the piston assembly 740 moves freely and with little friction relative to the provider housing 738. Alternatively, seals (not shown) can be used in one or more of the gaps 766, 768F, 768I, 768S.

The fluid source 736 is in fluid communication with the fluid inlets 754F, 754S. With this design, the fluid source 736 can selectively direct pressurized fluid 770 (illustrated as circles) to the fluid inlets 754F, 754S, respectively and into the intermediate chambers 764FI, 764SI, respectively and regulate the pressures in the intermediate chambers 764FI, 764SI. The fluid source 736 can be controlled by the control system 224 (illustrated in FIG. 2A).

FIGS. 7C-7G each illustrate a cross-sectional view of a force provider 732 and a simplified illustration of a mover 730 coupled to a stage 706, a fluid source 736, and a device 700. FIGS. 7C-7G illustrate movement of a center of gravity 771 (c.g.) of the stage 706 by the mover 730 and the force provider 732 along a stage path 772 that includes a first stage region 772F, a second stage region 772S, and a third stage region 772T. In FIG. 7C, the c.g. 771 of the stage 706 is in the second stage region 772S and approaching the first stage region 772F; in FIG. 7D, the c.g. 771 of the stage 706 is in the first stage region 772F; in FIG. 7E, the c.g. 771 of the stage 706 is in the first stage region 772F; in FIG. 7F, the c.g. 771 of the stage 706 is in the second stage region 772S; and in FIG. 7G, the c.g. 771 of the stage 706 is in the third stage region 772T.

In one embodiment, in the first stage region 772F and the third stage region 772T, the mover 730 and the force provider 732 provide an acceleration/deceleration force on the stage 706 that accelerates and decelerates the stage 706, and in the second stage region 772S, the mover 730 moves the stage 706 at a constant velocity. In this embodiment, the first stage region 772F and the third stage region 772T are also referred to as acceleration/deceleration regions, and the second stage region 772S is also referred to a constant velocity region. In one embodiment, processing of the device 700 occurs while the stage 706 and the device 700 are moved at constant velocity in the second stage region 772S.

It should be noted that the control system 24 (illustrated in FIG. 1) controls the mover 730 to precisely position and move the stage 706 back and forth along the entire stage path 772. One movement of the stage 706 along the stage path 772 is described below. Starting with the stage 706 in the constant velocity region 772S (illustrated in FIG. 7C) moving right to left along the stage path 772, at this time the mover 730 controls the trajectory of the stage 706 so that the stage 706 is moved at constant velocity. Once the c.g. 771 of the stage 706 enters the first stage region 772F (illustrated in FIGS. 7D), the mover 730 and the force provider 732 act in parallel to decelerate the stage 706. When the stage 706 is at the left end of the stage path 772 (illustrated in FIG. 7E), the stage 706 will be stopped by the mover 730. Subsequently, the stage 706 is accelerated from left to right along the stage path 772 by the mover 730 and the force provider 732. When the c.g. 771 of the stage 706 enters the constant velocity region 772S (illustrated in FIGS. 7C and 7F) moving left to right, at this time the mover 730 controls the trajectory of the stage 706 so that the stage 706 is moved at constant velocity. Once the c.g. 771 of the stage 706 enters the third stage region 772T (illustrated in FIG. 7G), the mover 730 and the force provider 732 act in parallel to decelerate the stage 706. When the stage 706 is at the right end of the stage path 772, the stage 706 will be stopped by the mover 730. Subsequently, the stage 706 is accelerated from right to left along the stage path 772 by the mover 730 and the force provider 732. Subsequently, the c.g. 771 of the stage 706 enters the constant velocity region 772S (illustrated in FIGS. 7C and 7F) moving right to left. In this embodiment, the mover 730 always controls the trajectory of the stage 706 so that the stage 706 follows the desired trajectory. This procedure can be repeated for motion of the stage 706 along the Y axis.

FIGS. 7C-G also illustrate the operation of the force provider 732 during this time. In this embodiment, the piston 756 moves relative to the provider housing 738 along a piston path 762 that includes a first piston region 762F, a second piston region 762S, and a third piston region 762T. In FIGS. 7C and 7F, the piston 756 is in the second piston region 762S; in FIGS. 7D and 7E, the piston 756 is in the first piston region 762F; and in FIG. 7G, the piston 756 is in the third piston region 762T. In this embodiment, (i) the piston 756 is in the first piston region 762F when the c.g. 771 of the stage 706 is in the first stage region 772F; (ii) the piston 756 is in the second piston region 762S when the c.g. 771 of the stage 706 is in the second stage region 772S; and (iii) the piston 756 is in the third piston region 762T when the c.g. 771 of the stage 706 is in the third stage region 772T. In this embodiment, the size of the regions 762F-762T can be changed by changing the location of the cylinder apertures 750F, 750S and the pistons.

When the piston 756 is the first piston region 762F, (i) the piston 756 is positioned between the first cylinder aperture 750F and the first intermediate piston 759A, (ii) the first intermediate piston 759A is positioned between the piston 756 and the first side wall 748F, and (iii) the second intermediate piston 759C is positioned between the second cylinder aperture 750S and the second side wall 748S. Further, the piston 756 and the first intermediate piston 759A can move concurrently for at least a portion of the time when the piston 756 is in the first piston region 762F and the piston 756 moves relative to the second intermediate piston 759C and the provider housing 738.

When the piston 756 is the second piston region 762S, (i) the piston 756 is positioned between the cylinder apertures 750F, 750S, (ii) the first intermediate piston 759A is positioned between the first cylinder aperture 750F and the first side wall 748F, and (iii) the second intermediate piston 759C is positioned between the second cylinder aperture 750S and the second side wall 748S. Further, the piston 756 moves independently and relative to the intermediate pistons 759A, 759C and the provider housing 738 when the piston 756 is the second piston region 762S.

When the piston 756 is the third piston region 762T, (i) the piston 756 is positioned between the second cylinder aperture 750S and the second intermediate piston 759C, (ii) the second intermediate piston 759C is positioned between the piston 756 and the second side wall 748S, and (iii) the first intermediate piston 759A is positioned between the first cylinder aperture 750F and the first side wall 748F. Further, the piston 756 and the second intermediate piston 759C can move concurrently for at least a portion of the time when the piston 756 is in the third piston region 762T and the piston 756 moves relative to the first intermediate piston 759A and the provider housing 738.

In one embodiment, when the piston 756 is in the first piston region 762F and in the third piston region 762T, the force provider 732 provides an acceleration/deceleration force on the stage 706, and in the second piston region 762S, the force provider 732 exerts substantially no force on the stage 706 and the stage 706 moves at a constant velocity. In this embodiment, the first piston region 762F and the third piston region 762T are also referred to as acceleration/deceleration regions, and the second piston region 762S is also referred to a constant velocity region.

One back and forth movement of the piston 756 along the piston path 762 is described below. Starting with the piston 756 in the constant velocity region 762S (illustrated in FIG. 7C) moving right to left along the piston path 762, the piston 756 is between the cylinder apertures 750F, 750S and the pressure on both sides of the piston 756 is approximately equal. At this time, the piston 756 is moved by the mover 730 along with the stage 706. Because the pressure is approximately equal on both sides of the piston 756 at this time, approximately no force will be acting on the piston 756. This minimizes transmissibility between the force provider 732 and the stage 706. At all times, the mover 730 controls the trajectory of the stage 706.

Referring to FIG. 7D, once the piston 756 enters the first piston region 762F on the left of the first cylinder aperture 750F, the force provider 732 acts in parallel with the mover 730 to decelerate the stage 706. More specifically, with the piston 756 moving to the left entering the first piston region 762F, the mover 730 starts providing force to decelerate the stage 706. At the same time, the piston 756 passes the first cylinder aperture 750F and the volume of fluid (e.g. air) between the piston 756 and the first intermediate piston 759A (the first chamber 764F) will start compressing and the pressure on the first piston side 760F is greater than the pressure on the second piston side 760S.

This creates a resultant pressure that is derived from the equation PV=nRT; where P is the absolute pressure in the closed system, V is the volume of this closed system, n is the unit of air, R is the gas constant and T is the temperature. Assuming that no air is leaking through the wall gaps 766, n will be constant. In addition, also assume that temperature T is constant. Then P∝1/V. The resultant force provided by the forcer provider 732 would just be equal to the area of the piston 756 times P (gauge pressure). Since the volume V decreases as the piston 756 moves to the left, the resultant force increases, adding a deceleration force from the force provider 732 in addition to the deceleration force provided by the mover 730. As a result, the peak force achievable for deceleration will be higher than with the mover 730 alone. The force output is a function of the compressed volume.

Initially, referring to FIG. 7D, when the piston 756 enters the first piston region 762F (just left of the first cylinder aperture 750F), the regulated pressure in the first intermediate chamber 764FI is greater than the pressure of the compressing fluid in the first chamber 764F. At this time the piston 756 is moving to the left relative to the first intermediate piston 759A and the first intermediate piston 759A is stationary. The fluid in the first chamber 764F will continue to compress as long as the pressure in the first chamber 764F is less than the pressure in the first intermediate chamber 764FI.

It should be noted because of the first intermediate piston 759A, the original volume of fluid in the first chamber 764F to be compressed is reduced. The smaller volume will result in a more rapid rise in pressure in the first chamber 764F as the piston 756 is moved towards the first intermediate piston 759A in the first piston region 762F. Additionally, it should be noted that the volume of fluid to be compressed in the first chamber 764F and the deceleration/acceleration characteristics can be adjusted by adjusting the initial position of the first intermediate piston 759A and the initial piston gap between the pistons 756, 759A when the piston 756 enters the first piston region 762F.

Eventually, referring to FIG. 7E, the pressure in the first chamber 764F will become slightly larger than the regulated pressure in the first intermediate chamber 764FI. At this time, the piston 756 and the first intermediate piston 759A will move concurrently from right to left. It should be noted the regulated pressure in the first intermediate chamber 764FI is controlled by the fluid source 736 and can be adjusted to achieve the desired forces on the piston 756.

Subsequently, the stage 706 will come to a complete stop. At this point in time, the mover 730 will still be providing force in the same direction, but the stage 706 would now start to accelerate to the right along the stage path 772. Meanwhile, the positive pressure built up on the first piston side 760F will still be adding an acceleration force from the force provider 732 to the force output from the mover 730. Thus, the stage 706 is accelerated from left to right along the stage path 772 by the mover 730 and the force provider 732. Gradually, the pressure in the first chamber 764F will fall below the pressure in the first intermediate chamber 764FI. When, the stage 706 enters the constant velocity region 772S (see FIG. 7C) moving left to right (see FIG. 7F), at this time the mover 730 controls the trajectory of the stage 706 so that the stage 706 is moved at constant velocity. At this time, the piston 756 is in the second piston region 762S and the pressure on both sides of the piston 756 is equal.

Once the stage 706 enters the third stage region 772T, the piston 756 is in the third piston region 762T, the mover 730 and the force provider 732 act in parallel to decelerate the stage 706. At this time, the piston 756 passes the second cylinder aperture 750S and the volume of air to the right of the second cylinder aperture 750S and the left of the second intermediate piston 759C (the second chamber 764S) will start compressing and the pressure on the second piston side 760S is greater than the pressure on the first piston side 760F. This results in a deceleration force from the force provider 732 on the stage 706.

Initially, when the piston 756 enters the third piston region 762T Oust right of the second cylinder aperture 750S), the regulated pressure in the second intermediate chamber 764SI is greater than the pressure of the compressed fluid in the second chamber 764S. At this time the piston 756 is moving to the right relative to the second intermediate piston 759C and the second intermediate piston 759C is stationary. The fluid in the second chamber 764S will continue to compress as long as the pressure in the second chamber 764S is less than the pressure in the second intermediate chamber 764SI.

It should be noted because of the second intermediate piston 759S, the original volume of fluid in the second chamber 764S to be compressed is reduced. The smaller volume will result in a more rapid rise in pressure in the second chamber 764S as the piston 756 is moved towards the second intermediate piston 759C in the third piston region 762T. Additionally, it should be noted that the volume of fluid to be compressed in the second chamber 764S and the deceleration/acceleration characteristics can be adjusted by adjusting the initial position of the second intermediate piston 759C and the initial piston gap between the pistons 756, 759C when the piston 756 enters the third piston region 762T.

Eventually, referring to FIG. 7G, the pressure in the second chamber 764S will become slightly larger than the regulated pressure in the second intermediate chamber 764SI. At this time, the piston 756 and the second intermediate piston 759C will move concurrently from left to right. It should be noted the regulated pressure in the second intermediate chamber 764SI is controlled by the fluid source 736 and can be adjusted to achieve the desired forces on the piston 756.

When the stage 706 is at the right end of the stage path 772, the stage 706 will be stopped by the mover 730. Subsequently, the stage 706 is accelerated from right to left along the stage path 772 by the mover 730 and the force provider 732. When, the stage 706 enters the constant velocity region 772S (illustrated in FIG. 7B) moving right to left, at this time the mover 730 controls the trajectory of the stage 706 so that the stage 706 is moved at constant velocity. This procedure can be repeated for motion of the stage 706 along the Y axis.

In one embodiment, the force provider 732 cannot be used alone and has no capability of position control and the force provider 732 provides force in a position where the volume has been compressed. In this embodiment, the force provider 732 is not actively controlled and a gauge pressure of zero is measured at each cylinder aperture 750F, 750S when the piston 756 is in the second piston region 762S.

In one embodiment, the fluid source 736 compensates for (i) fluid lost from the first intermediate chamber 764FI when the piston 756 is in the first piston region 762F and (ii) fluid lost from the second intermediate chamber 764SI when the piston 756 is in the third piston region 762T. For example, in one embodiment, (i) when the piston 756 is in the first piston region 762F, the amount of fluid directed into first fluid inlet 754F by the fluid source 736 is approximately equal to the amount of fluid that escapes from the first intermediate chamber 764FI; (ii) when the piston 756 is in the third piston region 762T, the amount of fluid directed into the second fluid inlet 754S by the fluid source 736 is approximately equal to the amount of fluid that escapes from the second intermediate chamber 764SI; and (iii) the fluid source 736 does not direct fluid into the fluid inlets 754F, 754S when the piston 756 is in the second piston region 762S.

In another embodiment, the fluid source 736 directs fluid into the first fluid inlet 754F so that the pressure on the first side 761A of the first intermediate piston 759A does not decrease when the piston 756 is in the first piston region 762F and the fluid source 736 directs fluid into the second fluid inlet 754S so that the pressure on the second side 763B of the second intermediate piston 759C does not decrease when the piston 756 is in the third piston region 762T.

In another embodiment, the amount of fluid loss when the piston 756 is in the first piston region 762F and/or the third piston region 762T is empirically calculated and the control system controls the fluid source 736 to compensate for the fluid loss. In alternative embodiments, the fluid source 736 directs fluid to the fluid inlets 754F, 754S at a rate of approximately 0.5, 1, 2, 3, 4 or 5 liters/minute.

In another embodiment, the force provider 732 provides dampening in addition or alternatively to an acceleration/deceleration force. This is accomplished by slowly leaking fluid when the piston 756 is in the first piston region 762F or the third piston region 762T.

In an alternative embodiment, for example, the first intermediate beam 759B can be replaced with another structure, such as a cable or spring that inhibits the first intermediate piston 759A from being moved too far away from the first side wall 748F and/or the second intermediate beam 759D can be replaced with another structure, such as a cable or spring that inhibits the second intermediate piston 759C from being moved too far away from the second side wall 748S.

FIGS. 7H is a cross-sectional view of still another embodiment of a force provider assembly 728H that can be used in the stage assembly 220A, 220B illustrated in FIG. 2A, FIG. 2B or another type of stage assembly. In FIG. 7H, the force provider assembly 728H includes a force provider 732H and a fluid source 736H that are similar to the corresponding components described above and illustrated in FIG. 7B.

However, in this embodiment, the piston assembly 740H includes a first channel 741F that extends into the first chamber 764FH and a second channel 741S that extends into the second chamber 764SH. The channels 741F, 741S can be connected to a fluid source (not shown) to adjust and/or control the pressure, or replace fluid loss through gaps, in the respective chambers 764FH, 764SH. Alternatively, the channels 741F, 741S can be connected to a gauge so that the pressure in the respective chamber 764FH, 764SH can be monitored. Still alternatively, the channels 741F, 741S can be connected to a valve that allows fluid in the respective chamber 764FH, 764SH to be selectively released. The location of the channels 741F, 741S can vary. In FIG. 7H, the first channel 741F extends through the first beam 758FH and the second channel 741S extends through the second intermediate beam 759DH and the second intermediate piston 759CH.

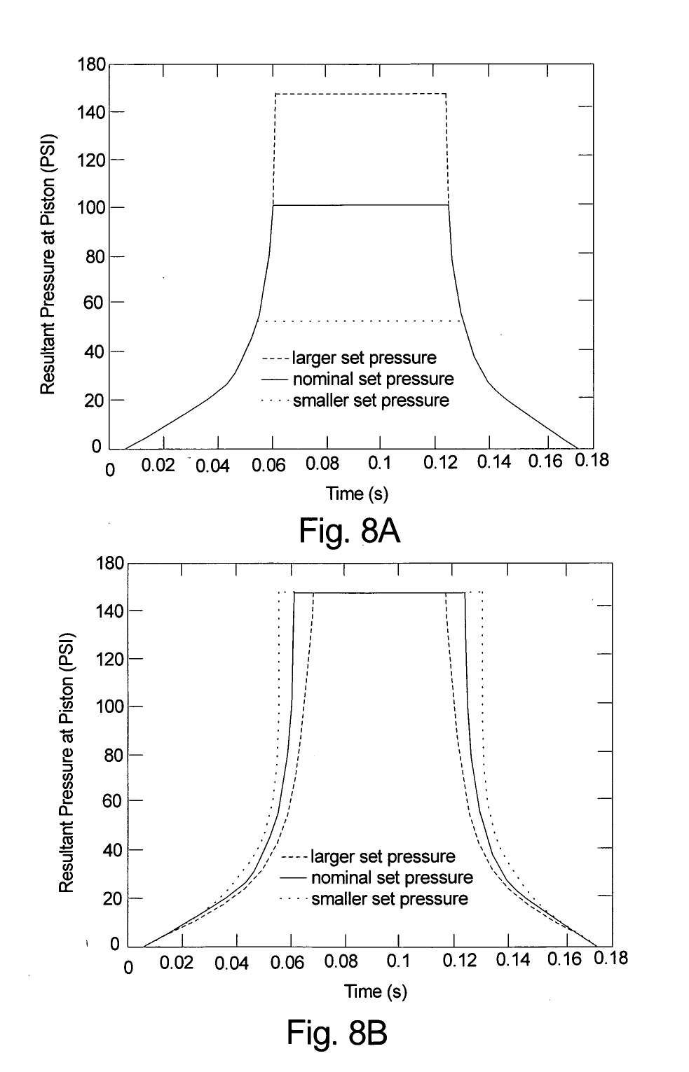

FIG. 8A is a graph that illustrates the pressure on the piston 756 when the piston is in one of the acceleration/deceleration regions 762F, 762T. More specifically, three separate lines illustrate how three separate set pressures influence pressure on the piston 756.

FIG. 8B is a graph that illustrates the influence of the piston gap on the pressure exerted on the piston 756 when the piston is in one of the acceleration/deceleration regions 762F, 762T. It should be noted that the pressure on the piston 756 increases and decreases more slowly as the piston gap is increased and pressure on the piston 756 increases and decreases more rapidly as the piston gap is decreased.

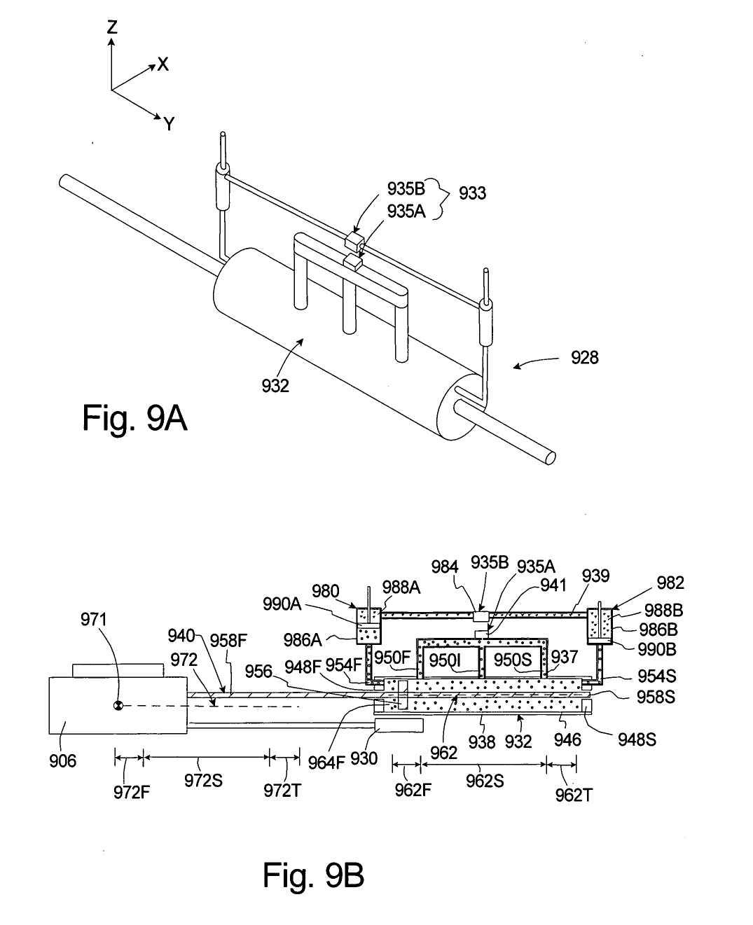

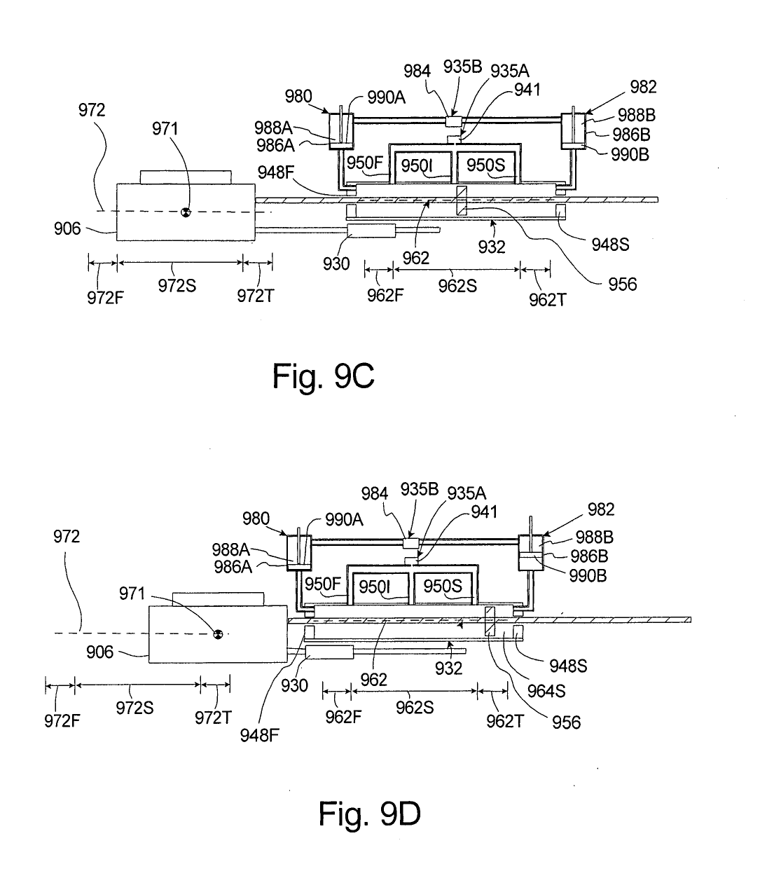

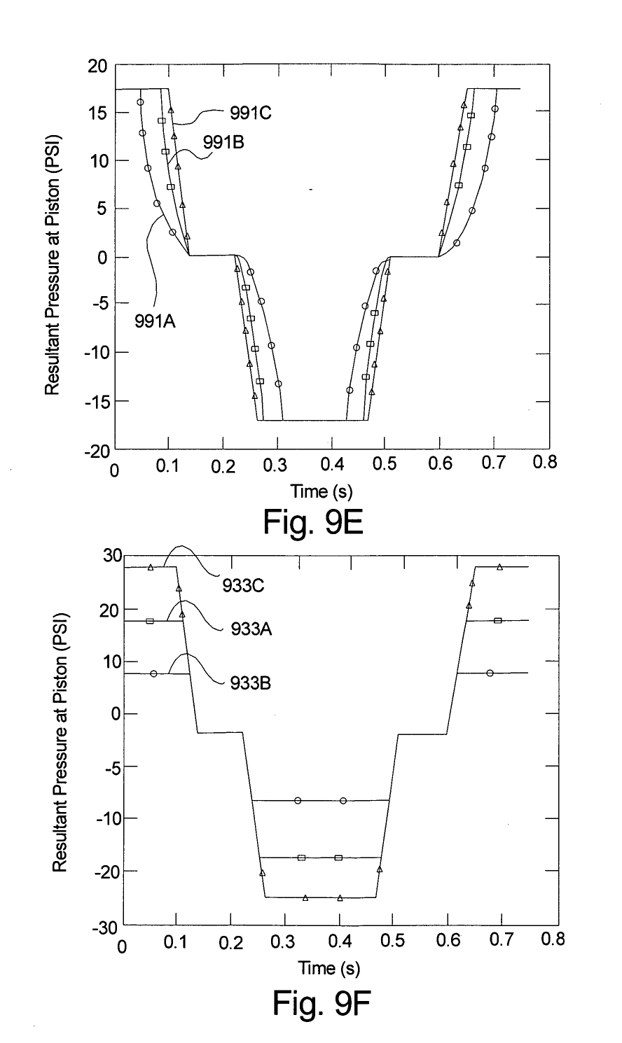

FIGS. 9A is a simplified perspective view of still another embodiment of a force provider assembly 928 that can be used in the stage assembly 220A, 220B illustrated in FIG. 2A, FIG. 2B or another type of stage assembly. Alternatively, the force provider assembly 928 can be used in another type of system to move or position another type of device or object during a manufacturing, measurement and/or inspection process. In FIG. 9A, the force provider assembly 928 includes a force provider 932, and a control assembly 933 that can adjust and control an acceleration/deceleration profile of the force provider 932. In one embodiment the control assembly 933 includes a first pressure control assembly 935A, and a second pressure control assembly 935B. Alternatively, for example, the force provider and/or the control assembly can be designed differently.

It should be noted that the control assembly 933 illustrated in FIG. 9A can be adapted to be used with the other force providers described above and illustrated herein.

FIG. 9B is a cross-sectional view of the force provider 932 and the pressure control assemblies 935A, 935B of FIG. 9A, and a simplified view of a stage 906 and a mover 930. In this embodiment, the force provider 932 includes a provider housing 938 and a piston assembly 940 that are somewhat similar to the corresponding components described above and illustrated in FIGS. 3A and 3B.

The provider housing 938 includes a cylinder wall 946, a disk shaped first side wall 948F, and a disk shaped second side wall 948S. The cylinder wall 946 includes a first cylinder aperture 950F, a spaced apart, second cylinder aperture 950S, and an intermediate cylinder aperture 950I that extend transversely through the cylinder wall 946. One or more of the cylinder apertures 950F, 950S, 950I are in fluid communication with the first pressure control assembly 935A. In FIG. 9B each of the cylinder apertures 950F, 950S, 950I is in fluid communication with the first pressure control assembly 935A. Alternatively, the provider housing can include more than three or less than three cylinder apertures.

In FIG. 9B, the first side wall 948F includes a first fluid inlet 954F that is in indirect fluid communication with the second pressure control assembly 935B across first PC piston 990A. Similarly, the second side wall 948S includes a second fluid inlet 954S that is in indirect fluid communication with the second pressure control assembly 935B across second PC piston 990B. Alternatively, for example, the fluid inlets 954F, 954S could be at another location, such as through the cylinder wall 946 near each end.

The piston assembly 940 includes a piston 956, a rigid first beam 958F and a rigid second beam 958S that are somewhat similar to the corresponding components described above and illustrated in FIGS. 3A and 3B. When the piston 956 is to the left of the first cylinder aperture 950F, the piston 956 cooperates with the cylinder wall 946 and the first side wall 948F to define a first chamber 964F, and when the piston 956 is to the right of the second cylinder aperture 950S the piston 956 cooperates with the cylinder wall 946 and the second side wall 948S to define a second chamber 964S (illustrated in FIG. 9D).

FIGS. 9B-9D illustrate movement of a center of gravity 971 (c.g.) of the stage 906 by the mover 930 and the force provider 932 along a stage path 972 that includes a first stage region 972F, a second stage region 972S, and a third stage region 972T. In this embodiment, in the first stage region 972F and the third stage region 972T, the mover 930 and the force provider 932 provide an acceleration/deceleration force on the stage 906 that accelerates and decelerates the stage 906, and in the second stage region 972S, the mover 930 moves the stage 906 at a constant velocity.

The control system 224 (illustrated in FIG. 2A) controls the mover 930 to precisely position and move the stage 906 back and forth along the stage path 972. An example of the movement of the stage 906 along the stage path 972 is described above during the discussion of FIGS. 4A-4C.

Additionally, FIGS. 9B-9D illustrate the operation of the force provider 932. In this embodiment, the piston 956 moves relative to the provider housing 938 along the piston path 962 that includes a first piston region 962F, a second piston region 962S, and a third piston region 962T. In FIG. 9B, the piston 956 is in the first piston region 962F, in FIG. 9C, the piston 956 is in the second piston region 962S, and in FIG. 9D, the piston 956 is in the third piston region 962T. In this embodiment, the size of the regions 962F-962T can be changed by changing the location of the first and second cylinder apertures 950F, 950S.

In the first piston region 962F, the piston 956 is positioned between the first side wall 948F and the first cylinder aperture 950F. In the second piston region 962S, the piston 956 is positioned between the first cylinder aperture 950F and second cylinder aperture 950S. In the third piston region 962T, the piston 956 is positioned between the second cylinder aperture 950S and the second side wall 948S.

When the piston 956 is in the first piston region 962F and in the third piston region 962T, the force provider 932 provides an acceleration/deceleration force on the stage 906, and in the second piston region 962S, the force provider 932 exerts substantially no force on the stage 906 and the stage 906 moves at a constant velocity. In this embodiment, the first piston region 962F and the third piston region 962T are also referred to as acceleration/deceleration regions, and the second piston region 962S is also referred to a constant velocity region.

The pressure control assemblies 935A, 935B can be used to adjust and precisely achieve the desired acceleration/deceleration characteristics of the force provider assembly 928. For example, the first pressure control assembly 935A can be used to control the ramping characteristics of the acceleration/deceleration profile and the second pressure control assembly 935B can be used to control the maximum force exerted by the force provider assembly 928.

In one embodiment, the first pressure control assembly 935A controls the pressure at the cylinder apertures 950F, 950S, 950I so that the cylinder apertures 950F, 950S, 950I are each at a first pressure. The actual value of the first pressure can be varied to achieve the desired performance characteristics of the force provider assembly 928. With certain designs, the value of the first pressure determines the ramping characteristics of acceleration/deceleration profile of the force provider assembly 928. Generally speaking, as value of the first pressure is increased, the rate at which the force generated by the force provider assembly 928 is increased. Further, as the value of the first pressure is decreased, the rate at which the force generated by the force provider assembly 928 is decreased.

In one embodiment, the first pressure is maintained at a pressure of between approximately 0 and 100 PSI. Stated another way, for example, in alternative non-exclusive embodiments, the first pressure is maintained at approximately 0, 5, 10, 20, 40, 80, or 100 PSI.

The design of the first pressure control assembly 935A can vary. In one embodiment, the first pressure control assembly 935A includes a first regulator 941 that regulates the pressure of a first fluid 937 (illustrated as circles in FIG. 9B) in the cylinder apertures 950F, 950S, 950I. The first regulator 941 can be actively controlled by the control system 224 (illustrated in FIG. 2A) or passively controlled. In the case of active controlling, feedback and feed forward control can be applied to the control of the first regulator 941. It should be noted that the first pressure control assembly 935A can include multiple regulators. Further, for example, the first pressure control assembly can include a container (not shown) of the first fluid and/or a fluid pump (not shown).

The maximum, second pressure control assembly 935B can be used to control the maximum force exerted by the force provider assembly 928. Stated in another fashion, the second pressure control assembly 935B can be used to control the maximum pressure in the first chamber 964F (when the piston 956 is to the left of the first cylinder aperture 950F) and the maximum pressure in the second chamber 964S (when the piston 956 is to the right of the second cylinder aperture 950S). In one embodiment, the second pressure control assembly 935B controls the maximum pressure at the fluid inlets 954F, 954S.

The actual maximum pressure can be varied to achieve the desired performance characteristics of the force provider assembly 928. With certain designs, the value of the maximum pressure determines the maximum force provided by the force provider assembly 928. Generally speaking, for a given first pressure, as value of the maximum pressure is increased, the value of the maximum force generated by the force provider assembly 928 is increased. Further, as the value of the maximum pressure is decreased, the value of the maximum force generated by the force provider assembly 928 is decreased.

In one embodiment, the maximum pressure is maintained at a pressure of between approximately 0 and 200 PSI. Stated another way, for example, in alternative non-exclusive embodiments, the maximum pressure is maintained at approximately 0, 20, 40, 80, 150, or 200 PSI. In certain embodiments, the maximum pressure is greater than the first pressure. For example, in alternative non-exclusive embodiments, the maximum pressure is approximately 5, 10, 20, 40, 80, or 100 PSI greater than the first pressure. Stated another way, for example, in alternative non-exclusive embodiments, the maximum pressure is approximately 20, 50, 100, 200, 400, or 1000 percent greater than the first pressure.

The design of the second pressure control assembly 935B can vary. In one embodiment, the second pressure control assembly 935B includes a first subassembly 980, a second subassembly 982, and a PC regulator 984. In this embodiment, the first subassembly 980 includes a first PC housing 986A that defines a PC chamber 988A, and a first PC piston 990A that moves in the PC chamber 988A relative to the PC housing 986A, and the second subassembly 982 includes a second PC housing 986B that defines a PC chamber 988B, and a second PC piston 990B that moves in the PC chamber 988B relative to the PC housing 986B.

For the first subassembly 980, in this embodiment, the pressure on the bottom first side of the first PC piston 990A is equal to the pressure in the first fluid inlet 954F. Similarly, for the second subassembly 982, in this embodiment, the pressure on the bottom first side of the second PC piston 990B is equal to the pressure in the second fluid inlet 954S. The PC regulator 984 regulates the pressure on the top second side of each PC piston 990A, 990B. With this design, the PC regulator 984 can control the maximum pressure. The PC regulator 984 can be actively controlled by the control system 224 (illustrated in FIG. 2A) or passively controlled. In the case of active controlling, feedback and feed forward control can be applied to the control of the PC regulator 984. It should be noted that the second pressure control assembly 935B can include more that two or less than two subassemblies.

Each PC chamber 988A, 988B can be filled with a fluid 939 (illustrated as triangles in FIG. 9B) at the desired pressure. The PC regulator 984 can include a fluid container (not shown) with the fluid or a fluid pump (not shown).

One back and forth movement of the piston 956 along the piston path 962 is described below. Starting with the piston 956 in the constant velocity region 962S (illustrated in FIG. 9C) moving right to left along the piston path 962, the piston 956 is between the first and second cylinder apertures 950F, 950S and the pressure on both sides of the piston 956 is approximately equal to the first pressure controlled by the first pressure control assembly 935A. Further the pressure on the first side of each PC piston 990A, 990B is also equal to the first pressure.

Once the piston 956 enters the first piston region 962F on the left of the first cylinder aperture 950F, the force provider 932 acts in parallel with the mover 930 to decelerate the stage 906. As the piston 956 passes the first cylinder aperture 950F, the volume of fluid to the left of the first cylinder aperture 950F will start compressing and the pressure on the left side of the piston 956 is greater than the pressure on the right side of the piston 956. The volume of fluid will continue to compress until the pressure in the first chamber 964F is equal to the pressure in the first PC chamber 988A above the first PC piston 990A. Subsequently, the first PC piston 990A begins to move and the pressure in the first PC chamber 988A stays close to the pressure regulated by second pressure control assembly 935B. With this design, the first subassembly 980 is able to regulate the maximum pressure experienced by the piston 956 and the maximum force generated by the force provider 932.

When, the stage 906 enters the constant velocity region 972S (illustrated in FIG. 9C) moving left to right, at this time the mover 930 controls the trajectory of the stage 906 so that the stage 906 is moved at constant velocity. At this time, the piston 956 is in the second piston region 962S and the pressure on both sides of the piston 956 is equal to the first pressure. Referring to FIG. 9D, once the stage 906 enters the third stage region 972T, the piston 956 is in the third piston region 962T, the mover 930 and the force provider 932 act in parallel to decelerate the stage 906. At this time, the piston 956 passes the second cylinder aperture 950S and the volume of air to the right of the second cylinder aperture 950S will start compressing and the pressure on the right side of the piston 956 is greater than the pressure on the left side of the piston 956. The volume of fluid will continue to compress until the pressure in the second chamber 964S is equal to the pressure in the second PC chamber 988B above the second PC piston 990B. Subsequently, the second PC piston 990B begins to move and the pressure in the second PC chamber 988B stays close to the pressure regulated by second pressure control assembly 935B. With this design, the second subassembly 982 is able to regulate the maximum pressure experienced by the piston 956 and the maximum force generated by the force provider 932.

FIG. 9E is a graph that illustrates the resultant pressure at the piston versus time during one back and forth movement of the piston along the piston path from the first piston region to the third piston region and back. FIG. 9E also illustrates the influences of changing the first pressure on the characteristics of the force provider assembly. In FIG. 9E, (i) line 991A represents the resulting pressure profile when the first pressure is maintained at 14.7 PSI and the maximum pressure is maintained at 31.7 PSI, (ii) line 991B represents the resulting pressure profile when the first pressure is maintained at 5 PSI and the maximum pressure is maintained at 22 PSI, and (iii) line 991C represents the resulting pressure profile when the first pressure is maintained at 30 PSI and the maximum pressure is maintained at 47 PSI. In each example, the difference between the first pressure and the maximum pressure is 17 PSI. However, the larger the first pressure, the quicker the maximum resultant pressure is reached.

FIG. 9F is another graph that illustrates the resultant pressure at the piston versus time during one back and forth movement of the piston along the piston path from the first piston region to the third piston region and back. FIG. 9F also illustrates the influences of changing the maximum pressure on the characteristics of the force provider assembly. In FIG. 9F, (i) line 993A represents the resulting pressure profile when the first pressure is maintained at 30 PSI and the maximum pressure is maintained at 47 PSI, (ii) line 993B represents the resulting pressure profile when the first pressure is maintained at 30 PSI and the maximum pressure is maintained at 37 PSI, and (iii) line 993C represents the resulting pressure profile when the first pressure is maintained at 30 PSI and the maximum pressure is maintained at 57 PSI. In this embodiment, the maximum pressure is greater as the difference between the first pressure and the maximum pressure is increased.

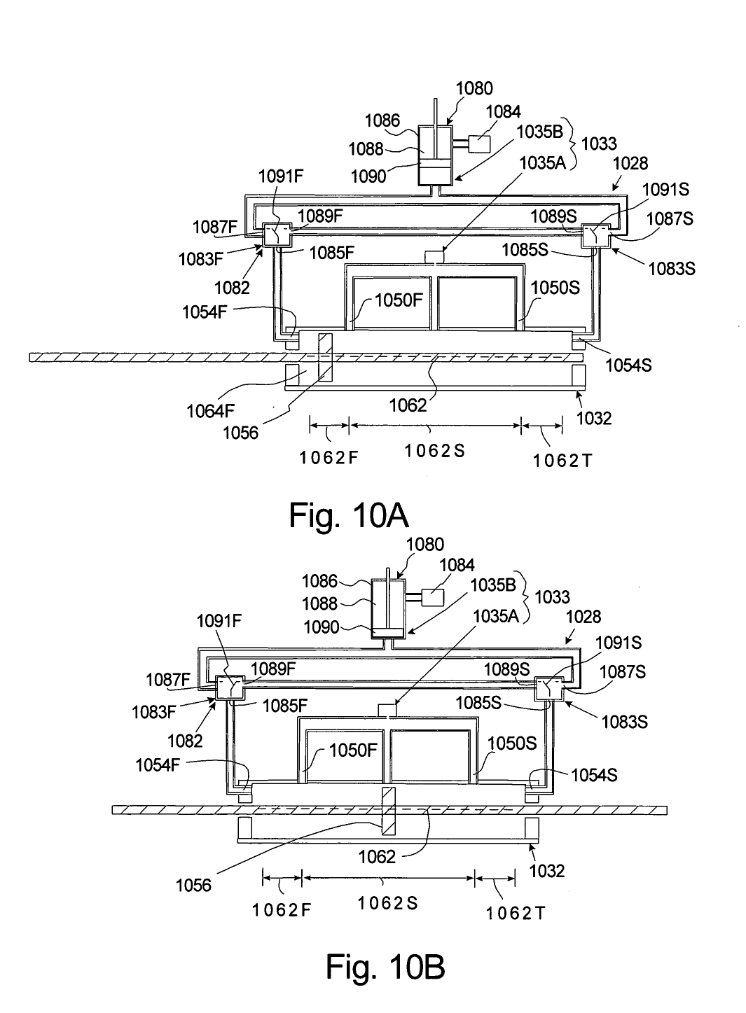

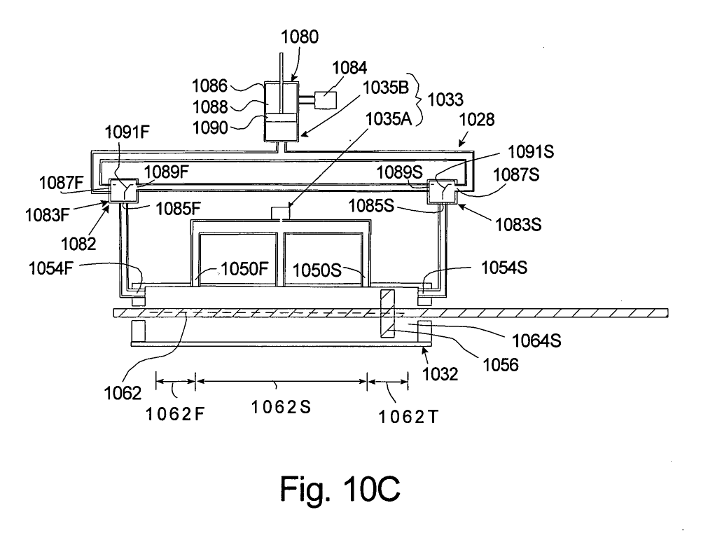

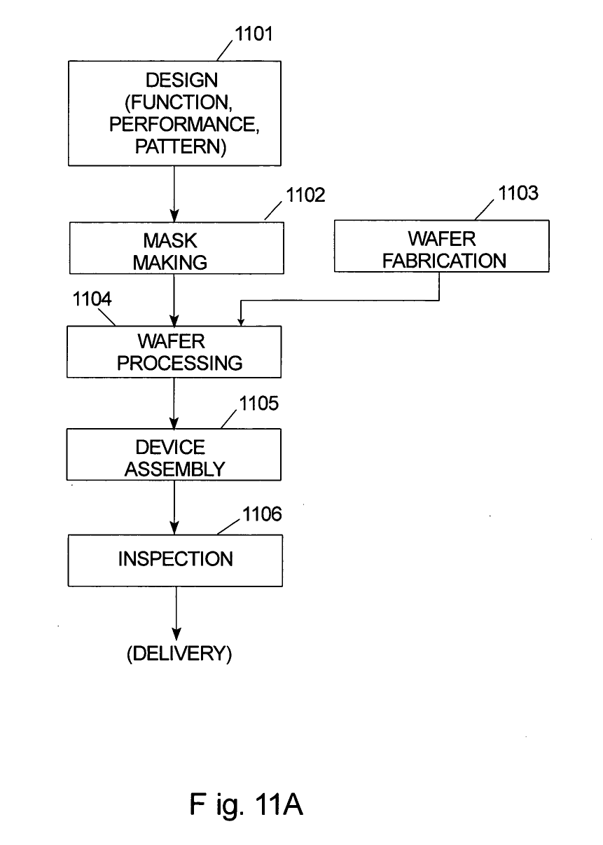

FIGS. 10A-10C are cross-sectional views that illustrate yet another embodiment of a force provider assembly 1028 that can be used in the stage assembly 220A, 220B illustrated in FIG. 2A, FIG. 2B or another type of stage assembly. Alternatively, the force provider assembly 1028 can be used in another type of system to move or position another type of device or object during a manufacturing, measurement and/or inspection process. In this embodiment, the force provider assembly 1028 includes a force provider 1032, and a control assembly 1033 having a first pressure control assembly 1035A and a second pressure control assembly 1035B that can adjust and control an acceleration/deceleration profile of the force provider 1032. Further, in this embodiment, the force provider 1032 and the first pressure control assembly 1035A are similar to the corresponding components described above and illustrated in FIGS. 9A-9D.

The second pressure control assembly 1035B is slightly different than the corresponding component described above. In the embodiment illustrated in FIGS. 10A-10C, the second pressure control assembly 1035B includes a PC piston assembly 1080, a PC flow switch assembly 1082, and a PC regulator 1084. In this embodiment, the PC piston assembly 1080 includes a PC housing 1086 that defines a PC chamber 1088, and a PC piston 1090 that moves in the PC chamber 1088 relative to the PC housing 1086.

Depending upon the position of the PC flow switch assembly 1082, the pressure on the bottom first side of the assembly piston 1090 is equal to the pressure in the first fluid inlet 1054F or the second fluid inlet 1054S. The PC regulator 1084 regulates the pressure on the back second side of the PC piston 1090. With this design, the PC regulator 1084 can control the maximum pressure. The PC regulator 1084 can be actively controlled by the control system 224 (illustrated in FIG. 2A) or passively controlled. In the case of active controlling, feedback and feed forward control can be applied to the control of the PC regulator 1084.

The PC flow switch assembly 1082 allows the PC piston assembly 1080 to independently be in fluid communication with the first fluid inlet 1054F and the second fluid inlet 1054S. In one embodiment, the PC flow switch assembly 1082 includes a first flow switch 1083F and a second flow switch 1083S. The first flow switch 1083F is used to selectively connect the first fluid inlet 1054F in fluid communication with the PC piston assembly 1080, and the second flow switch is used to selectively connect the second fluid inlet 1054S in fluid communication with the PC piston assembly 1080. In one embodiment, each of the flow switches 1083F, 1083S is a two way, electronic flow valve. The flow switches 1083F, 1083S can be actively controlled by the control system 224 (illustrated in FIG. 2A).

In one embodiment, the first flow switch 1083F includes (i) an inlet 1085F that is connected in fluid communication with the first fluid inlet 1054F with a conduit, (ii) a first outlet 1087F that is connected in fluid communication with the PC piston assembly 1080 with a conduit, (iii) a second outlet 1089F that is connected in fluid communication with the second flow switch 1083S with a conduit, and (iv) a valve 1091F that is moved to selectively connect the inlet 1085F in fluid communication with the first outlet 1087F or the second outlet 1089F. Similarly, the second flow switch 1083S includes (i) an inlet 1085S that is connected in fluid communication with the second fluid inlet 1054S with a conduit, (ii) a first outlet 1087S that is connected in fluid communication with the PC piston assembly 1080 with a conduit, (iii) a second outlet 1089S that is connected in fluid communication with the first flow switch 1083F with a conduit, and (iv) a valve 1091S that is moved to selectively connect the inlet 1085S in fluid communication with the first outlet 1087S or the second outlet 1089S.

One back and forth movement of the piston 1056 along the piston path 1062 is described below. Starting with the piston 1056 in the Constant velocity region 1062S (illustrated in FIG. 10B) moving right to left along the piston path 1062, the piston 1056 is between the first and second cylinder apertures 1050F, 1050S and the pressure on both sides of the piston 1056 is approximately equal to the first pressure controlled by the first pressure control assembly 1035A.

Once the piston 1056 enters the first piston region 1062F on the left of the first cylinder aperture 1050F, the force provider 1032 acts in parallel with the mover (illustrated in FIG. 9B) to decelerate the stage (illustrated in FIG. 9B). As the piston 1056 passes the first cylinder aperture 1050F, the first flow switch causes the first fluid inlet 1054F to be in fluid communication with the PC piston assembly 1080, the second flow switch causes the second fluid inlet 1054S to not be in fluid communication with the PC piston assembly 1080, the volume of fluid to the left of the first cylinder aperture 1050F will start compressing and the pressure on the left side of the piston 1056 is greater than the pressure on the right side of the piston 1056. The volume of fluid will continue to compress until the pressure in the first chamber 1064F is equal to the pressure in the PC chamber 1088 above the PC piston 1090. Subsequently, the PC piston 1090 begins to move and the pressure in the PC chamber 1088 begins to compress.

When, the stage enters the constant velocity region moving left to right, at this time the mover controls the trajectory of the stage so that the stage is moved at constant velocity. Referring to FIG. 10C, once the piston 1056 is in the third piston region 1062T, the piston 1056 passes the second cylinder aperture 1050S, the second flow switch 1083S causes the second fluid inlet 1054S to be in fluid communication with the PC piston assembly 1080, the first flow switch causes the first fluid inlet 1054F to not be in fluid communication with the PC piston assembly 1080, and the volume of air to the right of the second cylinder aperture 1050S will start compressing and the pressure on the right side of the piston 1056 is greater than the pressure on the left side of the piston 1056. The volume of fluid will continue to compress until the pressure in the second chamber 1064S is equal to the pressure in the assembly chamber 1088 above the assembly piston 1090. Subsequently, the assembly piston 1090 begins to move and the pressure in the assembly chamber 1088 begins to compress.

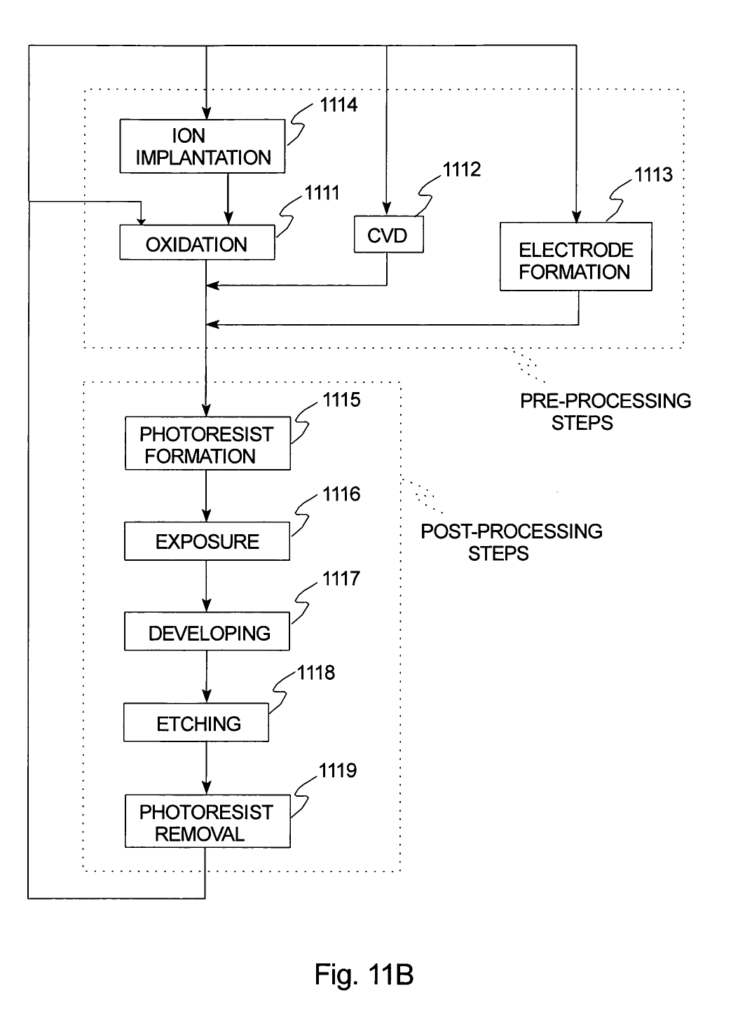

Semiconductor devices can be fabricated using the above described systems, by the process shown generally in FIG. 11A. In step 1101 the device's function and performance characteristics are designed. Next, in step 1102, a mask (reticle) having a pattern is designed according to the previous designing step, and in a parallel step 1103 a wafer is made from a silicon material. The mask pattern designed in step 1102 is exposed onto the wafer from step 1103 in step 1104 by a photolithography system described hereinabove in accordance with the present invention. In step 1105, the semiconductor device is assembled (including the dicing process, bonding process and packaging process), finally, the device is then inspected in step 1106.

FIG. 11B illustrates a detailed flowchart example of the above-mentioned step 1104 in the case of fabricating semiconductor devices. In FIG. 11B, in step 1111 (oxidation step), the wafer surface is oxidized. In step 1112 (CVD step), an insulation film is formed on the wafer surface. In step 1113 (electrode formation step), electrodes are formed on the wafer by vapor deposition. In step 1114 (ion implantation step), ions are implanted in the wafer. The above mentioned steps 1111-1114 form the preprocessing steps for wafers during wafer processing, and selection is made at each step according to processing requirements.

At each stage of wafer processing, when the above-mentioned preprocessing steps have been completed, the following post-processing steps are implemented. During post-processing, first, in step 1115 (photoresist formation step), photoresist is applied to a wafer. Next, in step 1116 (exposure step), the above-mentioned exposure device is used to transfer the circuit pattern of a mask (reticle) to a wafer. Then in step 1117 (developing step), the exposed wafer is developed, and in step 1118 (etching step), parts other than residual photoresist (exposed material surface) are removed by etching. In step 1119 (photoresist removal step), unnecessary photoresist remaining after etching is removed.

Multiple circuit patterns are formed by repetition of these preprocessing and post-processing steps.

While the particular force provider assembly 228A as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A force provider assembly comprising:
a provider housing that defines a piston chamber, the provider housing including a first beam aperture, a first cylinder aperture, and a spaced apart second cylinder aperture;
a piston assembly including a piston positioned in the piston chamber, and a first beam extending through the first beam aperture, the piston including a first piston side and a second piston side, the first beam being secured to the first piston side, the piston moving relative to the provider housing along a piston path, wherein at a first piston region of the piston path, the piston is positioned between the first beam aperture and the first cylinder aperture and at a second piston region of the piston path, the piston is positioned between the cylinder apertures; and
a first pressure control assembly that is in fluid communication with the cylinder apertures, the first pressure control assembly controlling the pressure in each cylinder aperture so that the pressure at each cylinder aperture is approximately the same.

2. The force provider assembly of claim 1 wherein the provider housing includes a second beam aperture, the piston assembly includes a second beam extending through the second beam aperture, the second beam being secured to the second piston side.

3. The force provider assembly of claim 1 wherein at the first piston region, the pressure of the fluid on the first piston side is greater than the pressure of the fluid on the second piston side.

4. The force provider assembly of claim 3 wherein at the second piston region, the pressure of the fluid on the first piston side is approximately equal to the pressure of the fluid on the second piston side.

5. The force provider assembly of claim 4 wherein at a third piston region of the piston path, the pressure of the fluid on the second piston side is greater than the pressure of the fluid on the first piston side and wherein in the third piston region, the piston is positioned between the second cylinder aperture and a second beam aperture in the provider housing.

6. The force provider assembly of claim 1 wherein the first pressure control assembly includes a regulator that controls the pressure at each cylinder aperture.

7. The force provider assembly of claim 1 further comprising a maximum pressure control assembly that is in fluid communication with a first fluid inlet in the provider housing, the maximum pressure control assembly controlling the maximum pressure at the first fluid inlet when the piston is in the first piston region.

8. The force provider assembly of claim 7 wherein the maximum pressure control assembly includes a PC housing, and a PC piston that is positioned within and that moves relative to the PC housing, and wherein a first piston side of the PC piston is in fluid communication with the first fluid inlet.

9. The force provider assembly of claim 8 wherein the maximum pressure control assembly includes a PC regulator that regulates a pressure on a second piston side of the PC piston.

10. A stage assembly for moving a device along a stage path that includes a first stage region and a second stage region, the stage assembly comprising:
a stage that retains the device;
a mover that moves the stage along the stage path; and
the force provider assembly of claim 1 coupled to the stage.

11. The stage assembly of claim 10 wherein the force provider assembly provides an acceleration/deceleration force on the stage when the stage is in the first stage region and approximately no force on the stage when the stage is in the second stage region.

12. The stage assembly of claim 11 wherein the stage path includes a third stage region and the force provider provides an acceleration/deceleration force on the stage when the stage is in the third stage region.

13. An exposure apparatus including the stage assembly of claim 10.

14. A process for manufacturing a device that includes the steps of providing a substrate and forming an image to the substrate with the exposure apparatus of claim 13.

15. A process for manufacturing a wafer that includes the steps of providing a substrate and forming an image to the substrate with the exposure apparatus of claim 13.

16. A force provider assembly comprising:
a provider housing that defines a piston chamber, the provider housing including a first beam aperture, a first cylinder aperture, a spaced apart second cylinder aperture, and a first fluid inlet;
a piston assembly including a piston positioned in the piston chamber, and a first beam extending through the first beam aperture, the piston including a first piston side and a second piston side, the first beam being secured to the first piston side, the piston moving relative to the provider housing along a piston path, wherein at a first piston region of the piston path, the piston is positioned between the first fluid inlet and the first cylinder aperture and at a second piston region of the piston path, the piston is positioned between the cylinder apertures; and
a maximum pressure control assembly that is in fluid communication with the first fluid inlet, the maximum pressure control assembly controlling the maximum pressure at the first fluid inlet when the piston is in the first piston region.

17. The force provider assembly of claim 16 wherein the maximum pressure control assembly includes a PC housing and a PC piston that is positioned within and that moves relative to the PC housing, and wherein a first piston side of the PC piston is in fluid communication with the first fluid inlet.

18. The force provider assembly of claim 17 wherein the maximum pressure control assembly includes a PC regulator that regulates a pressure on a second piston side of the PC piston.

19. A stage assembly for moving a device along a stage path that includes a first stage region and a second stage region, the stage assembly comprising:
a stage that retains the device;
a mover that moves the stage along the stage path; and
the force provider assembly of claim 16 coupled to the stage.

20. The stage assembly of claim 19 wherein the force provider assembly provides an acceleration/deceleration force on the stage when the stage is in the first stage region and approximately no force on the stage when the stage is in the second stage region.

21. The stage assembly of claim 20 wherein the stage path includes a third stage region and the force provider provides an acceleration/deceleration force on the stage when the stage is in the third stage region.

22. An exposure apparatus including the stage assembly of claim 19.

23. A process for manufacturing a device that includes the steps of providing a substrate and forming an image to the substrate with the exposure apparatus of claim 22.

24. A process for manufacturing a wafer that includes the steps of providing a substrate and forming an image to the substrate with the exposure apparatus of claim 22.

25. A force provider assembly for use with a mover for moving a stage along a stage path that includes a first stage region and a second stage region, the force provider assembly comprising:
a pneumatic force provider coupled to the stage, the force provider providing an acceleration/deceleration force on the stage when the stage is in the first stage region and approximately no force on the stage when the stage is in the second stage region, the pneumatic force provider including a control assembly that controls the acceleration/deceleration force.

26. The force provider assembly of claim 25 wherein the stage path includes a third stage region and the pneumatic force provider provides an acceleration/deceleration force on the stage when the stage is in the third stage region.

27. The force provider assembly of claim 25 wherein the control assembly controls the ramping characteristics of the acceleration/deceleration force.

28. The force provider assembly of claim 25 wherein the control assembly controls the maximum force of the acceleration/deceleration force.

29. The force provider assembly of claim 25 wherein the control assembly controls the ramping characteristics of the acceleration/deceleration force and the maximum force of the acceleration/deceleration force.

30. A force provider assembly of claim 25 wherein the force provider comprises (i) a provider housing that defines a piston chamber, the provider housing including a first beam aperture, a first cylinder aperture that is in fluid communication with a fluid at a first pressure and a spaced apart second cylinder aperture that is in fluid communication with a fluid at approximately the first pressure; and (ii) a piston assembly including a piston positioned in the piston chamber, and a first beam extending through the first beam aperture, the piston including a first piston side and a second piston side, the first beam being secured to the first piston side, the piston moving relative to the provider housing along a piston path, wherein at a first piston region of the piston path, the piston is positioned between the first beam aperture and the first cylinder aperture and at a second piston region of the piston path, the piston is positioned between the cylinder apertures.

31. A method for moving a stage along a stage path that includes a first stage region and a second stage region, the method comprising the steps of:
coupling a mover to the stage that moves the stage along the stage path; and
coupling a pneumatic force provider assembly to the stage, the force provider providing an acceleration/deceleration force on the stage when the stage is in the first stage region and approximately no force on the stage when the stage is in the second stage region, the pneumatic force provider including a control assembly that controls the acceleration/deceleration force.

32. The method of claim 31 wherein the stage path includes a third stage region and the pneumatic force provider provides an acceleration/deceleration force on the stage when the stage is in the third stage region.

33. The method of claim 31 wherein the control assembly controls the ramping characteristics of the acceleration/deceleration force.

34. The method of claim 31 wherein the control assembly controls the maximum force of the acceleration/deceleration force.

35. The method of claim 31 wherein the control assembly controls the ramping characteristics of the acceleration/deceleration force and the maximum force of the acceleration/deceleration force.

36. A method for making an exposure apparatus that forms an image on a wafer, the method comprising the steps of:
providing an irradiation apparatus that irradiates the wafer with radiation to form the image on the wafer; and
providing a stage by the method of claim 31.

37. A method of making a wafer utilizing the exposure apparatus made by the method of claim 36.
